SEQUENCE LISTING (15 sequences)

SEQ ID NO: 1 - DNA, 15 bp, Artificial Sequence
  Description of Artificial Sequence Synthetic oligonucleotide
rgnacannntgtncy

SEQ ID NO: 2 - DNA, 6 bp, Artificial Sequence
  Description of Artificial Sequence Synthetic oligonucleotide
rgnaca

SEQ ID NO: 3 - DNA, 10 bp, Artificial Sequence
  Description of Artificial Sequence Synthetic oligonucleotide
cwnnntgtnc

SEQ ID NO: 4 - DNA, 22 bp, Artificial Sequence
  Description of Artificial Sequence Synthetic primer
tgcaaagagcaagacagaaaag

SEQ ID NO: 5 - DNA, 22 bp, Artificial Sequence
  Description of Artificial Sequence Synthetic primer
gcggtcatagagacacaattcc

SEQ ID NO: 6 - DNA, 24 bp, Artificial Sequence
  Description of Artificial Sequence Synthetic primer
ggctgtctgagtgaatctgtagtg

SEQ ID NO: 7 - DNA, 22 bp, Artificial Sequence
  Description of Artificial Sequence Synthetic primer
agttacatttgcccttgattcc

SEQ ID NO: 8 - DNA, 23 bp, Artificial Sequence
  Description of Artificial Sequence Synthetic primer
ggagaaacagactttacctgacc

SEQ ID NO: 9 - DNA, 22 bp, Artificial Sequence
  Description of Artificial Sequence Synthetic primer
tgttgcagtcaaggagacagag

SEQ ID NO: 10 - DNA, 23 bp, Artificial Sequence
  Description of Artificial Sequence Synthetic primer
gcctgttcctattaagtgtcctg

SEQ ID NO: 11 - DNA, 25 bp, Artificial Sequence
  Description of Artificial Sequence Synthetic primer
ggctgtaaagttatttgctagattg

SEQ ID NO: 12 - DNA, 21 bp, Artificial Sequence
  Description of Artificial Sequence Synthetic primer
aacgtcctcttcctgctcttg

SEQ ID NO: 13 - DNA, 22 bp, Artificial Sequence
  Description of Artificial Sequence Synthetic primer
gagctttgatgtagacggaatg

SEQ ID NO: 14 - DNA, 20 bp, Artificial Sequence
  Description of Artificial Sequence Synthetic primer
ccgagcagcaataactttcc

SEQ ID NO: 15 - DNA, 22 bp, Artificial Sequence
  Description of Artificial Sequence Synthetic primer
acgcaagaacacagtgagtagc

(19) United States
(12) Patent Application Publication
(10) Pub. No.: US 2014/0363425 A1
(43) Pub. Date: Dec. 11, 2014

(54) SYSTEMS AND METHODS FOR IDENTIFYING CANCERS HAVING ACTIVATED PROGESTERONE RECEPTORS

(71) Applicants: J. DINNY GRAHAM, Fairlight (AU); CHRISTINE L. CLARKE, Faulconbridge (AU)

(72) Inventors: J. DINNY GRAHAM, Fairlight (AU); CHRISTINE L. CLARKE, Faulconbridge (AU)

(21) Appl. No.: 14/205,694

(22) Filed: Mar. 12, 2014

Related U.S. Application Data
(60) Provisional application No. 61/779,720, filed on Mar. 13, 2013.

(51) Int. Cl. G01N 33/53

(52) U.S. Cl. CPC G01N 33/5308
USPC 424/133.1; 514/179; 514/171

(57) ABSTRACT

Systems and methods for identifying tumors having activated progesterone receptors are provided. Patients suspected of having a tumor susceptible to growth inhibition by anti-progestins can be treated with an anti-progestin.

### PRIORITY CLAIM

This application claims priority to U.S. Provisional Patent Application Ser. No. 61/779,720, filed Mar. 13, 2013. The above referenced application is incorporated herein by reference as if restated in full.

### BACKGROUND

Considerable effort has been applied over several decades to understanding the molecular mechanisms of progesterone signaling in target tissues such as the breast and endometrium. Progesterone regulates transcription via its nuclear receptor (PR), which associates with specific target sites on chromatin. The consensus deoxyribonucleic acid (DNA) sequence to which PR binds (progesterone response element (PRE)) consists of six nucleic acid base pairs in an inverted repeated sequence: RGNACAnnnTGTNCY [1, 2, 3]. DNA-bound PR recruits transcriptional co-activators and associated co-factors, which modify the local chromatin structure and facilitate transcriptional activation, resulting in activation or repression of PR target genes [4, 5, 6, 7]. In addition to co-regulators and co-factors, which associate with the PR regulatory complex by protein-protein interaction, PR recruits chromatin remodeling factors, which modify local DNA architecture to enhance PR interaction and transcriptional activation [8]. Factors known to be involved in chromatin remodeling at progestin-regulated sites include the SWI/SNF chromatin remodeling complex [8, 9] and transcription factor NF1, which cooperates with PR for binding and activation of MMTV [10, 11]. For other nuclear receptors including estrogen receptor (ER) and androgen receptor (AR), pioneer factors such as FOXA1, which interact with condensed chromatin, are required for nuclear receptor activation of transcriptional targets [12, 13, 14, 15, 16, 17]. In addition to direct interaction with DNA at PREs, PR has been reported to associate with target genes via tethering to other transcription factors, including AP-1, SP1 and Stat3 [18, 19, 20, 21, 22].

Although determinants governing the transcriptional activity of PR have been described in vitro, the molecular basis for the pleomorphic roles for progesterone in vivo is poorly understood. Progesterone is needed for normal reproductive tissue function [23] and in the uterus supports differentiation, and inhibits proliferation of the endometrium [24]. By contrast, in the breast, progesterone is associated with increased proliferation, ductal side-branching and lobuloalveolar development [25]. Consistent with the distinct effects of progesterone in these two tissues, there are distinct transcriptional responses to progesterone in the breast and endometrium [23, 26, 27, 28, 29, 30].

Exposure to exogenous progestins in hormone replacement therapy is associated with increased breast cancer risk [31, 32, 33, 34, 35]. Interestingly, progestins regulate different transcriptomes in breast cancer cells compared with normal breast [36], so it is plausible that the effect of progestins on breast cancer risk may be mediated by altered specificity of progestin action in the precancerous and/or cancerous breast tissue. If altered cell-specificity of PR underlies the deleterious effect of progestins on breast cancer risk, the determinants of cell-specificity of progestin action require elucidation.

### SUMMARY

In one aspect, systems and methods for identifying activated progesterone receptor in various tissue types are provided. In another aspect, activated progesterone receptors in a tumor tissue are identified by detecting binding of the progesterone receptor to genomic (DNA) binding sites. These exemplary systems and methods can be used to identify and treat patients suspected of having a malignancy susceptible to growth inhibition and cancer cell apoptosis by anti-progestins (e.g., onapristone, lonaprisan, mifepristone, PF-02413873, telapristone, lilopristone, ORG2058, apoprisnil, ulipristal, ZM172406, ZM150271, ZM172405 and aglepristone). In one aspect, patients suspected of having a malignancy (cancer) susceptible to growth inhibition with anti-progestins can be treated with anti-progestins. In another aspect, cancers susceptible to treatment with anti-progestins include, but are not limited to, breast, brain, meningiomas, prostate, ovarian, endometrial, uterine sarcomas, uterine leiomyoma and lung. In a further aspect, the anti-progestin can be administered to a patient in an amount from about 10 mg to about 200 mg per day. Optionally, an anti-tumor compounds (e.g., everolimus, trastuzumab, TM1-D, anti-HER2 drugs, bevacizumab, paclitaxel, docetaxel, taxanes, doxorubicin, liposomal doxorubicin, pegylated liposomal doxorubicin, anthracyclines, anthracenediones, carboplatin, cisplatin, 5-FU, gemcitabine, cyclophosphamide, anti-estrogen, selective estrogen receptor modulators, aromatase inhibitors, and anti-androgens) may also be administered to the patient concurrently, before, or after treatment with the anti-progestin.

Aspects described herein provide methods and systems for identification of cancers and tumors susceptible to treatment with anti-progestins. In one aspect, activated progesterone receptor or PR bound to specific genomic DNA regions, as described herein, can be identified and quantified to identify malignancies susceptible to treatment with anti-progestins.

In another aspect, quantification of specific genomic DNA regions at a level that is, for example, at least about four-fold greater in the test sample than detection of the same specific region in a comparative negative control reference sample within the assay indicates the presence of activated PR. Exemplary comparative negative control samples for such quantitation (in one aspect, collectively referred to herein as “control DNA sequence”) include, but are not limited to, (1) isolated input genomic DNA from a pre-cleared sample, for example in a ChIP-seq assay, as described herein, and (2) a chromatin immunoprecipitation of a test sample in, for example, a ChIP-PCR assay, performed in the presence of a non-specific immunoglobulin or no primary antibody, and otherwise treated in the same manner as the specific PR chromatin immunoprecipitation of the test sample.

In yet another aspect, anti-progestins suitable for use herein include, but are not limited to:

Onapristone, (e.g., (8S,11R,13R,14S,17S)-11-[4-(dimethylamino)phenyl]-17-hydroxy-17-(3-hydroxypropyl)-13-methyl-1,2,6,7,8,11,12,14,15,16-decahydrocyclopenta[a]phenanthren-3-one) has the following chemical structure:

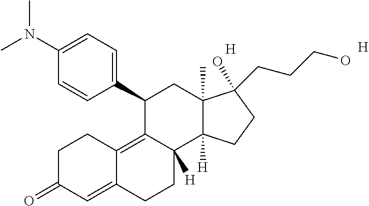

Other anti-progestins include: progestational 3-(6,6-ethylene-17B-hydroxy-3-oxo-17A-pregna-4-ene-17A-YL)propionic acid G-lactones, 3-(6,6-ethylene-17.beta.-hydroxy-3-oxo-17.alpha.-pregna-4-ene-17.alpha.-y-l)propionic acid .gamma.-lactone and the following:

Mifepristone (10S,11S,14S,15S,17R)-17-[4-(dimethylamino)phenyl]-14-hydroxy-15-methyl-14-(prop-1-yn-1-yl)tetracyclo [8.7.0.0̂{.2,7}.0̂{11,15}]heptadeca-1,6-dien-5-one

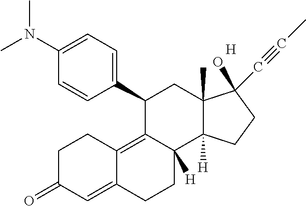

Lilopristone (11-beta,17-beta,17 (z))-ropenyl);estra-4,9-dien-3-one,11-(4-(dimethylamino)phenyl)-17-hydroxy-17-(3-hydroxy-1-p;11β-[4-(Dimethylamino)phenyl]-17β-hydroxy-17-[(Z)-3-hydroxy-1-propenyl]estra-4,9-dien-3-one

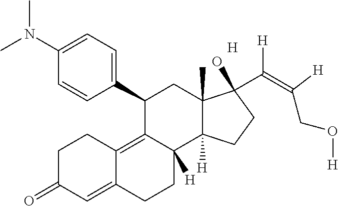

ORG2058 (8R,9S,10R,13S,14S,16R,17S)-16-ethyl-17-(2-hydroxyacetyl)-13-methyl-2,6,7,8,9,10,11,12,14,15,16,17-dodecahydro-1H-cyclopenta[a]phenanthren-3-one

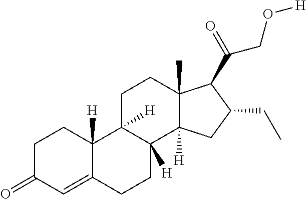

Lonaprisan (8S,11R,13S,14S,17S)-11-(4-acetylphenyl)-17-hydroxy-13-methyl-17-(1,1,2,2,2-pentafluoroethyl)-1,2,6,7,8,11,12,14,15,16-decahydrocyclopenta[a]phenanthren-3-one

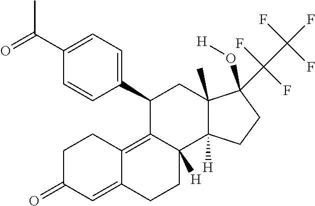 [SMILES: CC(=O)c1ccc([C@H]2C[C@@]3(C)[C@@H](CC[C@@]3(O)C(F)(F)C(F)(F)F)[C@@H]3CCC4=CC(=O)CCC4=C32)cc1]

Asoprisnil (8S,11R,13S,14S,17S)-11-[4-[(E)-hydroxyiminomethyl]phenyl]-17-methoxy-17-(methoxymethyl)-13-methyl-1,2,6,7,8,11,12,14,15,16-decahydrocyclopenta[a]phenanthren-3-one

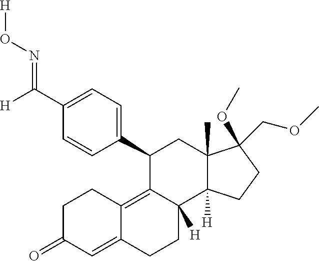 [SMILES: COC[C@]1(OC)CC[C@H]2[C@@H]3CCC4=CC(=O)CCC4=C3[C@@H](c3ccc(/C=N/O)cc3)C[C@@]21C]

Ulipristal (8S,11R,13S,14S,17R)-17-acetyl-11-[4-(dimethylamino)phenyl]-17-hydroxy-13-methyl-1,2,6,7,8,11,12,14,15,16-decahydrocyclopenta[a]phenanthren-3-one

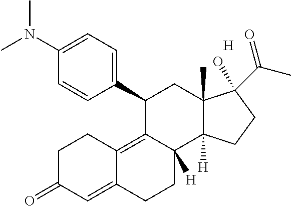

PF-2413873 4-[3-Cyclopropyl-1-(mesylmethyl)-5-methyl-1H-pyrazol-4-yl]oxy,-2,6-dimethylbenzonitrile

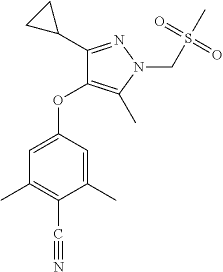 [SMILES: Cc1cc(Oc2c(C3CC3)nn(CS(C)(=O)=O)c2C)cc(C)c1C#N]

Aglepristone (8S,11R,13S,14S,17R)-11-(4-dimethylaminophenyl)-17-hydroxy-13-methyl-17-[(Z)-prop-1-enyl]-1,2,6,7,8,11,12,14,15,16-decahydrocyclopenta[a]phenanthren-3-one:

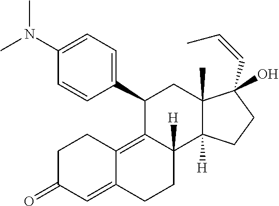

Additional anti-progestins include the following:

ZM172406—(R)—N-(3-chloro-4-cyanophenyl-3,3,3-trifluoro-2-hydroxy-2-methylpropanamide:

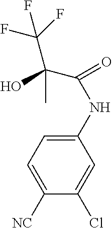 [SMILES: C[C@@](O)(C(=O)Nc1ccc(C#N)c(Cl)c1)C(F)(F)F]

ZM172405—(S)—N-(3-chloro-4-cyanophenyl)-3,3,3-trifluoro-2-hydroxy-2-methylpropanamide:

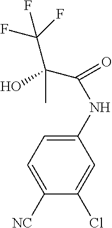 [SMILES: C[C@](O)(C(=O)Nc1ccc(C#N)c(Cl)c1)C(F)(F)F]

ZM150271—N-(3-chloro-4-cyanophenyl)-3,3,3,-trifluoro-2-hydroxy-2-methylpropanamide:

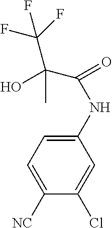 [SMILES: CC(O)(C(=O)Nc1ccc(C#N)c(Cl)c1)C(F)(F)F]

The transcriptional effects of the ovarian hormone progesterone are pleiotropic, and binding to DNA of the nuclear progesterone receptor (PR), a ligand-activated transcription factor, results in diverse outcomes in a range of target tissues. To determine the distinct patterns of genomic interaction of PR contributing to the cell specificity of the PR transcriptome, we compared the genomic binding sites for PR in breast cancer cells and immortalized normal breast cells. PR binding was correlated with transcriptional outcome in both cell lines, with 60% of progestin-regulated genes associated with one or more PR binding regions.

There was an unexpected low overlap between the PR cistromes of the two cell lines, and a similarly low overlap in transcriptional targets. A conserved PR binding element (region of DNA) was identified in PR DNA binding regions from both breast cancer and normal breast cell lines, but there were distinct patterns of enrichment of known cofactor binding motifs, with FOXA1 sites over-represented in breast cancer cell binding regions and NF1 and AP-1 motifs uniquely enriched in the immortalized normal line. Downstream analyses suggested that differential cofactor availability may generate these distinct PR cistromes (e.g., specific progesterone receptor binding sites on genomic DNA), indicating that the cofactor levels may modulate PR specificity to DNA binding regions. The cell-specificity of PR binding can be determined by the coordinated effects of key binding cofactors. This information can also be used to identify and treat patients suspected of having a tumor susceptible to growth inhibition by anti-progestins (e.g., onapristone, lonaprisan, mifepristone, PF-02413873, telapristone, lilopristone, ORG2058, apoprisnil, ulipristal, ZM172406, ZM150271, ZM172405 and aglepristone).

### FIGURES

FIGS. 1A and 1B illustrate the distribution of distances from the nearest gene transcription start site (TSS) for each of the PR binding regions in T-47D and AB32 cells as shown in FIG. 1A and 1B, respectively;

FIGS. 1C and 1D illustrate linear regression analysis of binding regions against gene number per chromosome in T-47D (FIG. 1C) and AB32 (FIG. 1D);

FIG. 1E illustrates the median distance of PR binding to up-regulated genes;

FIG. 1F illustrates binding region distribution with respect to up- and down-regulated genes in AB32 cells;

FIGS. 2A-F illustrate gene expression profiles in T-47D and AB32 cells determined at 2, 6, and 24 hours after treatment with 10 nM ORG2058;

FIG. 3A illustrates PR binding regions in common between T-47D and AB32 cells;

FIG. 3B illustrates transcript expression measured by whole genome microarray in T-47D and AB32 cells treated with 10 nM ORG2058 (ORG) or vehicle for 2, 6 and 24 hours;

FIG. 3C illustrates overlap between transcripts regulated by progestins in T-47D and AB32 cells at 2, 6 or 24 hours;

FIG. 3D illustrates numbers of progestin regulated transcripts in T-47D and AB32 at individual treatment times;

FIG. 4A illustrates full length and core PREs identified in binding regions in T-47D and AB32 cells;

FIGS. 4B-C illustrate PR binding region sequences analyzed for statistical enrichment of conserved sequence motifs using MEME-ChIP and Homer;

FIGS. 4D-E illustrate PRE classifications as strong, moderate, or weak/absent;

FIG. 5A illustrate unsupervised cluster analysis of transcriptional profiles in response to progestin in AB32 cells in the presence and absence of FOXA1;

FIG. 5B illustrates FOXA1 protein expression in AB32 cells and parent MCF-10A cells before and after viral transduction, compared with endogenous expression in T-47D cells;

FIG. 5C illustrates numbers of progestin regulated transcripts in AB32 cells in the presence and absence of FOXA1;

FIG. 5D illustrates a comparison of FOXA1 binding strength at PR, ER and FOXA1 binding sites;

FIG. 5E illustrates the ratio of PR binding regions to regulated genes in AB32 cells for genes that lost, gained or retained progestin regulation with FOXA1 expression;

FIG. 5F illustrates the distance from PR binding regions in AB32 to genes that lost, gained or retained progestin regulation with FOXA1 expression

FIG. 6 illustrates progesterone receptor (PR) binding in relation to chromosome distribution in T-47D and AB32 cells;

FIGS. 7A-B illustrate the relationship between PR binding and time of progestin regulation;

FIGS. 8A-B illustrate the location of PR binding regions;

FIG. 9 illustrates the patterns of transcriptional regulation in T-47D cells;

FIG. 10 illustrates patterns of transcriptional regulation in AB32 cells;

FIG. 11 illustrates PR binding regions in T-47D and AB32 cells;

FIGS. 12A-C illustrate the validation cell type-specific PR binding regions identified in ChIP-seq;

FIG. 13 illustrates the overlap of PR binding regions in ORG2058-treated T-47D and AB32 cells with binding in T-47D cells after progesterone (P4) treatment;

FIGS. 14A-B illustrate progestin regulation of gene expression in additional breast cell lines;

FIGS. 15A-B illustrate PRE and cofactor motif enrichment in regulation-associated binding sites in T-47D and AB32 cells;

FIGS. 16A-B illustrate the distribution of PRE position in PR binding regions in T-47D and AB32 cells;

FIGS. 17A-B illustrate that PRE strength does not predict PR binding;

FIG. 18 illustrates FOXA1 transcript expression in cell lines;

FIG. 19 illustrates FOXA1 binding at PR binding regions with or without predicted FOXA1 motifs; and

FIG. 20 illustrates PR expression in T-47D, AB32 and AB9 cells.

### DESCRIPTION

In one aspect, the DNA sequence of the response elements to which the PR binds, the availability of transcriptional cofactors, and the chromatin architecture of the target cell have a combined effect on the specificity of the PR transcriptome. In another aspect, the contribution of progesterone receptor (PR) response elements, transcriptional co-factors, and chromatin architecture in normal breast and breast cancer cells can be determined using genome-wide PR chromatin immunoprecipitation, coupled with high-throughput sequencing, to compare PR interaction on genomic DNA in an exemplary breast cancer cell line (T-47D) and immortalized normal breast cells which stably express both the PR-A and PR-B isoforms (AB32, a stable PR expressing clone of MCF-10A). In yet another aspect, exemplary PR cistromes are identified and characterized. These PR cistromes can be used, for example, to identify patients with malignancies (e.g., breast cancer, endometrial cancer, ovarian cancer, prostate cancer, lung cancer and uterine sarcomas) susceptible to treatment with anti-progestins as described herein. Identifying and treating patients susceptible to treatment with anti-progestins may impact cancer development, proliferation, growth and metastases in such patients.

The activity and state of the progesterone receptor in various normal and tumorigenic tissues can be predicted. See, e.g., U.S. patent application Ser. No. 13/644,872, incorporated by reference herein in its entirety. This information can be used to identify and treat patients suspected of having a tumor susceptible to growth inhibition by anti-progestins (e.g., onapristone, lonaprisan, mifepristone, PF-02413873, telapristone, lilopristone, ORG2058, apoprisnil, ulipristal, ZM172406, ZM150271, ZM172405 and aglepristone). In one aspect herein, we describe methods and systems for determining the activity and state of the progesterone receptor by binding of the progesterone receptor to genomic DNA targets in a tissue sample suspected of being tumorigenic or cancerous taken from a patient, detecting the transcription level of one or more genomic DNA targets compared to a control DNA sequence, and administering an anti-progestin to the patient if the transcription level of the one or more genomic DNA targets is at least about four-fold greater than the transcription level of the control DNA sequence.

In one aspect, the term “cistrome” refers to the set of DNA regions within the genome, which are bound by a specific cis-acting transcriptional regulator (e.g., PR).

As used herein, the term “activated progesterone receptor associated DNA target” refers to a region of genomic DNA to which the progesterone receptor is capable of binding and is associated with an increase in transcription of a progesterone receptor cistrome compared to a control region of genomic DNA.

The term “administer” refers to providing a drug or drugs, prescribing one or more drugs, or placing one or more drugs on a formulary. The term “providing” refers to dispensing the drug directly to patient through any suitable route of administration (e.g., oral, injection, intravenous, intramuscular, and transdermal etc.) or providing instructions to a patient to do the same.

The term “progestin” refers to a natural or synthetic progestational substance that mimics some or all of the actions of progesterone, also referred to as progesterone receptor modulators (PRM) or selective progesterone receptor modulators (SPRM).

The term “anti-progestin” refers to a substance that inhibits the formation, transport, or action of or inactivates progestational agents, including, but not limited to, onapristone, lonaprisan, mifepristone, PF-02413873, telapristone, lilopristone, ORG2058, apoprisnil, ulipristal, ZM172406, ZM150271, ZM172405 and aglepristone. A PRM or SPRM may have some anti-progestin properties, and be considered an anti-progestin or a progestin depending on the context of use.

The term “bind” or “binding” refers to an association of one or more moieties on a molecule or chemical compound through interactions or chemical bonds (e.g., hydrogen, hydrophobic, ionic, and covalent).

Methods and systems are provided herein for determining whether a patient is susceptible to treatment with anti-progestins. In one aspect, a method of treating a patient with an anti-progestin comprises obtaining a tissue sample suspected of being tumorigenic or cancerous from a patient, binding the progesterone receptor to genomic DNA in the tissue sample, detecting genomic DNA associated with the progesterone receptor in the tissue sample, detecting the level of one or more activated progesterone receptor associated DNA targets in the tissue sample and in a negative control sample and administering an anti-progestin to the patient if the level of the one or more activated progesterone receptor associated DNA targets is at least about 4-fold greater than the level in the negative control sample.

Tissue samples or biopsies can be obtained from a patient, for example, by a surgeon, physician, nurse, or medical technician from a patient suspected of having a tumor or presenting with symptoms of cancer or other abnormal cell growth.

The tissue sample can be treated or prepared for analysis by, for example, cross-linking the sample by exposure to 1% formaldehyde in phosphate buffered saline (PBS). The sample can be resuspended in lysis buffer and the genomic DNA can be fragmented by sonication.

Progesterone receptor (PR)-bound genomic DNA can be immunoprecipitated by incubation with an anti-PR primary antibody complexed with a secondary antibody-magnetic bead conjugate. In one aspect, the assay can be conducted using at least duplicate determinations and matched negative control incubations containing samples without the use of the primary antibody. The magnetic bead complexes can be washed with buffers to reduce the non-specific signal.

Genomic DNA can be eluted from the magnetic beads. The cross-links can be reversed by heating to, for example, 65° C. for at least four hours in the presence of high salt. PCR can be used to purify genomic DNA fragments.

Activated PR associated DNA targets can be detected by quantitative real-time PCR. In one aspect, the samples are amplified in triplicate. In another aspect, at least four activated PR associated DNA targets are selected.

Activated PR associated DNA target abundance in the test samples can be determined relative to negative control/reference samples incubated in the absence of specific primary antibody. In one aspect, fold difference=2̂−(Mean Ct^{test}−Mean Ct reference)). The statistical significance of the fold difference can be determined, for example, by Student's t test of unpaired replicate count determination. In one aspect, a fold difference of about four fold or greater and a p value of less than 0.05 indicates the presence of activated PR.

In another aspect, if activated PR is detected, an anti-progestin can be administered to the patient.

In yet another aspect, the tissue sample is selected from group consisting of breast, brain, meningiomas, prostate, ovarian, endometrial, uterine sarcomas, uterine leiomyoma and lung tissue.

In one aspect, the anti-progestin is selected from the group consisting of onapristone, lonaprisan, mifepristone, PF-02413873, telapristone, lilopristone, ORG2058, apoprisnil, ulipristal, ZM172406, ZM150271, ZM172405 and aglepristone.

In another aspect, the anti-progestin is administered to the patient in an amount from about 10 mg to about 200 mg per day.

In yet another aspect, an anti-tumor compound (e.g., everolimus, trastuzumab, TM1-D, anti-HER2 drugs, bevacizumab, paclitaxel, docetaxel, taxanes, doxorubicin, liposomal doxorubicin, pegylated liposomal doxorubicin, anthracyclines, anthracenediones, carboplatin, cisplatin, 5-FU, gemcitabine, cyclophosphamide, anti-estrogen, selective estrogen receptor modulators, aromatase inhibitors, and anti-androgens) in addition to an anti-progestin can be administered or co-administered to the patient.

In one aspect, the activated progesterone receptor associated DNA targets includes PR binding regions associated with or located near ACSL1 and PACSIN1. In another aspect, the presence of a co-factor binding motif (e.g., FOXA1) can also be detected.

In another aspect, immunoprecipitation can be performed using monoclonal antibodies hPRa6 and hPRa7.

Other aspects provide a system for determining the activation state of a progesterone receptor in a tissue sample. The system can include, for example, an anti-PR primary antibody and one or more nucleic acid probes directed to one or more activated progesterone receptor associated DNA targets.

In another aspect, the nucleic acid probes can be selected from the group of probes consisting of nucleic acid probes for detecting ACSL1 and PACSIN1.

In yet another aspect, the antibodies are selected from the group consisting of monoclonal antibodies hPRa6 and hPRa7.

Other aspects provide methods for inhibiting the growth of a tumor susceptible to growth inhibition by anti-progestins by obtaining a tissue sample suspected of being tumorigenic or cancerous from the tumor of a patient, binding the progesterone receptor to genomic DNA in the tissue sample, detecting genomic DNA associated with the progesterone receptor in the tissue sample, detecting the transcription level of one or more activated progesterone receptor associated DNA targets and a control DNA sequence in the tissue sample, and administering an anti-progestin to the patient if the transcription level of the one or more activated progesterone receptor associated DNA targets is at least about 4-fold greater than the transcription level of a control DNA sequence, wherein the growth of the tumor is inhibited.

In one aspect, the anti-progestin can be administered to the patient in an amount from about 10 mg to about 200 mg per day.

Before describing several exemplary aspects described herein, it is to be understood that the invention is not limited to the details of construction or process steps set forth in the following description. The aspects described herein are capable of being practiced or being carried out in various ways.

### EXAMPLES

### Example 1

### Generation of Genome-Wide PR Interaction Profiles

In one aspect, PR genomic interactions were mapped in T-47D breast cancer cells and in the AB32 cell line: a stable PR expressing clone of the MCF-10A immortalized normal breast cell line. Cells were treated with the progestin ORG2058 (10 nM, 45 minutes), followed by PR-chromatin immunoprecipitation (ChIP) and Illumina sequencing. Sequences were aligned to the human genome and genomic regions enriched in the alignments were identified using the Bowtie [37] and ERANGE [38] software tools (false discovery rate 0.27%).

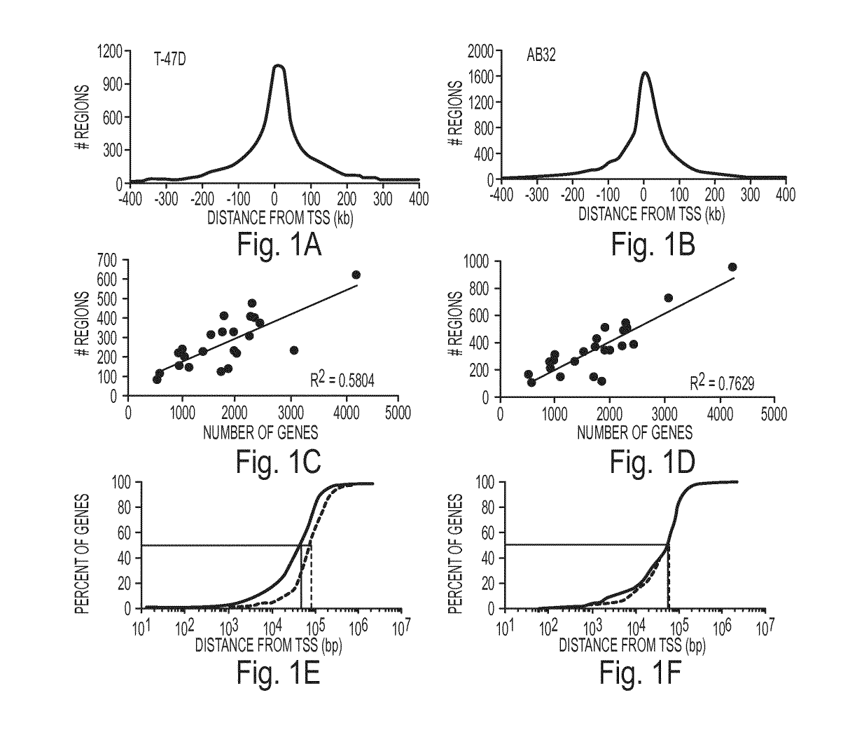

In T-47D cells, 6312 peaks of PR binding were identified and in AB32 cells 8117 binding regions were detected (Table 1). Most PR binding regions (88% in T-47D and 73% in AB32) were within 100 kb of the nearest gene, with 57% of binding regions in T-47D and 54% in AB32 within 50 kb. However, few binding regions (21% in T-47D and 20% in AB32) fell within 10 kb of a gene TSS (Table 1). The distribution of distances from the nearest gene transcription start site (TSS) for each of the PR binding regions in T-47D and AB32 cells is shown in FIGS. 1A and 1B, respectively.

PR binding regions were detected on all chromosomes and the number of binding sites per chromosome reflected chromosome size and number of genes on that particular chromosome, although some variability was observed. Linear regression analysis of binding regions against gene number per chromosome in T-47D (FIG. 1C) and AB32 (FIG. 1D) cells revealed a stronger correlation to gene number in the AB32 cells (R2=0.76 in AB32 compared to R2=0.58 in T-47D). Overall there was a correlation between numbers of binding regions per chromosome between the two cell lines (R2=0.66, Supplementary FIG. 1), however some exceptions were noted. Binding regions on chromosomes 2 and 8 were under-represented in the T-47D dataset compared to AB32, whereas regions on chromosome 11 were over-represented. The karyotype of T-47D cells [39] shows significant rearrangement and duplication compared to AB32 cells and this may partly explain the binding differences observed since T-47D cells contain 4 rather than 2 copies of chromosome 11. However, chromosome 2 is normal in T-47D cells, yet binding to regions on this chromosome were half as frequent as were detected in AB32 cells. Functional annotation of regulated genes on chromosome 2 that were bound by PR in AB32 revealed enrichment in genes involved in metabolism (Supplementary Table 1), suggesting an altered or attenuated metabolic response to progestins in the cancer cell line.

### Example 2

### Relationship Between PR Genomic Interaction and Transcriptional Response

In another aspect, gene expression profiling conducted in parallel with ChIP-seq revealed that PR binding regions were concentrated around regulated genes. The density of PR binding regions per gene, for example, was higher for regulated genes (density of binding regions per regulated gene: 2.23 in T-47D cells, 2.19 in AB32 cells; Table 1) than the overall PR binding region density (0.73 per gene for all genes in T-47D cells, 0.74 in AB32 cells; Table 1). In addition, PR binding peaks were more likely to be within 50 kb of the gene transcription start site in regulated genes (74% and 69% of regulated genes in T-47D and AB32 cells), compared with the proportion of PR binding regions within 50 kb of all genes (57% of PR binding regions within 50 kb of TSSs in T-47D cells, 54% in AB32 cells, Table 1).

PR binding regions in T-47D cells were on average closer to up-regulated gene TSSs than regions near down-regulated genes. In T-47D cells the median distance of PR binding to up-regulated genes was 44 kb, whereas median distance to down-regulated genes was 75 kb (FIG. 1E). This was reflected in a statistically significant overall difference in the cumulative frequency distributions of binding region distances to up-regulated and down-regulated genes in this cell line (FIG. 1E, p=0.001, Kolmogorov-Smirnov two-sample test). In contrast, no significant difference was seen in binding region distribution with respect to up- and down-regulated genes in AB32 cells (FIG. 1F, p=0.305).

In addition to PR binding regions being closer to up-regulated genes, there were more PR binding regions near up-regulated genes, for example, with an average 2.3 binding regions per up-regulated gene compared with 1.5 per down-regulated gene in T-47D cells and 2.4 and 1.9 average regions per up- and down-regulated gene, respectively, in AB32 cells (Table 1). Moreover, a higher proportion of up-regulated genes (509/786-65% of up-regulated genes in T-47D and 439/546-80% of up-regulated genes in AB32) were associated with PR binding regions than down-regulated genes (50/98-51% of down-regulated genes in T-47D and 325/621-52% of down-regulated genes in AB32, Table 1).

### Example 3

### PR Binding is Associated with Transcriptional Regulation

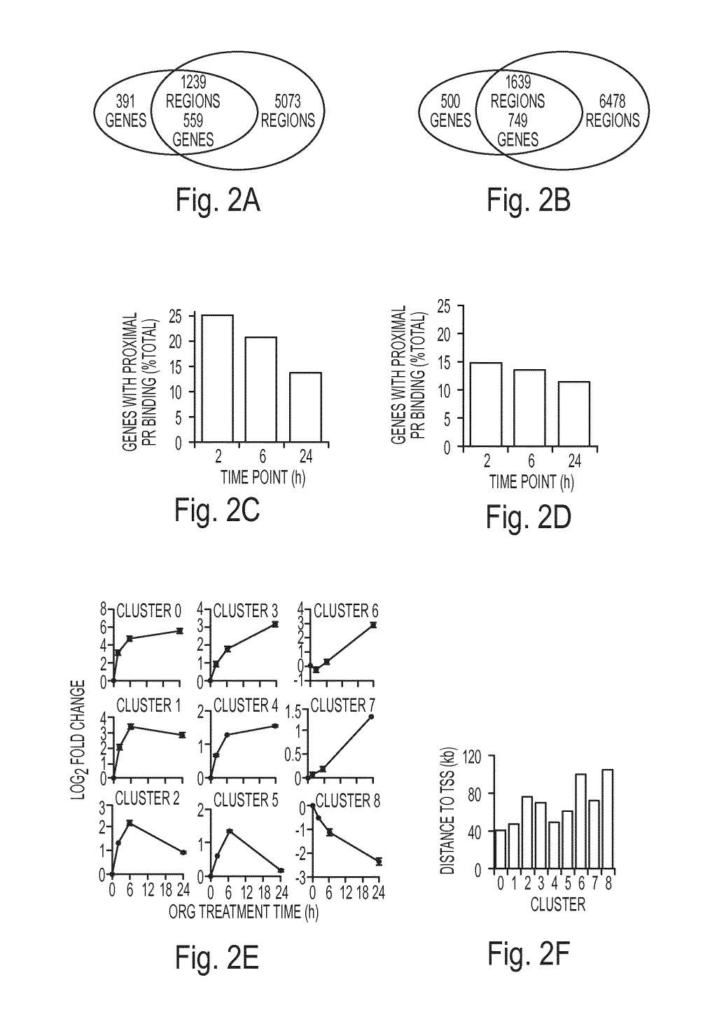

In another aspect, the majority of regulated genes in both cell lines (559/950 in T-47D (59%) and 749/1249 (60%) in AB32, FIGS. 2A and B, Table 1) had one or more PR binding region within 100 kb. There was a stronger association between PR binding and transcriptional regulation at earlier time points after ORG2058 treatment, suggesting that genes that are directly regulated by PR are more likely to be detected early at the transcriptional level than those that are indirect targets (Supplementary FIG. 2). This relationship was strongest in T-47D cells, and in AB32 cells was true only for binding regions that were relatively near the TSS of regulated genes, as shown by the higher representation of promoter proximal PR binding regions (5′UTR and up to 10 kb upstream) at earlier times in both cell lines (FIGS. 2C and D).

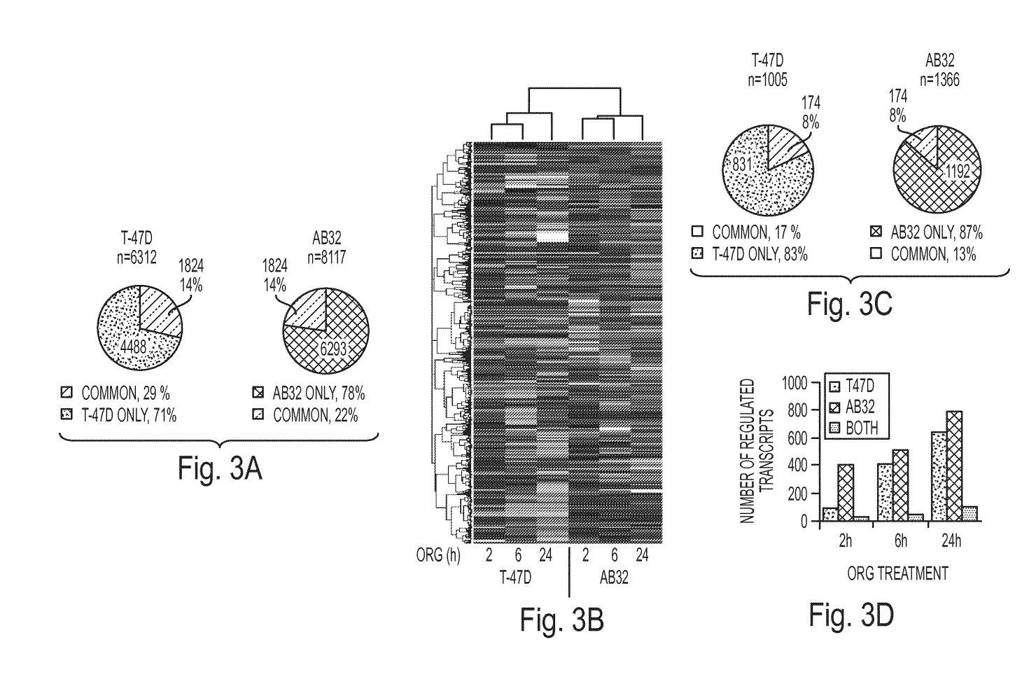

In another aspect, the overall distribution of PR binding regions with respect to intragenic and intergenic regions was similar in both cell lines (Supplementary FIG. 3): the greatest proportion of PR binding regions was observed upstream and in the 5′UTR of regulated genes, representing 43-45% of regions associated with regulated genes.

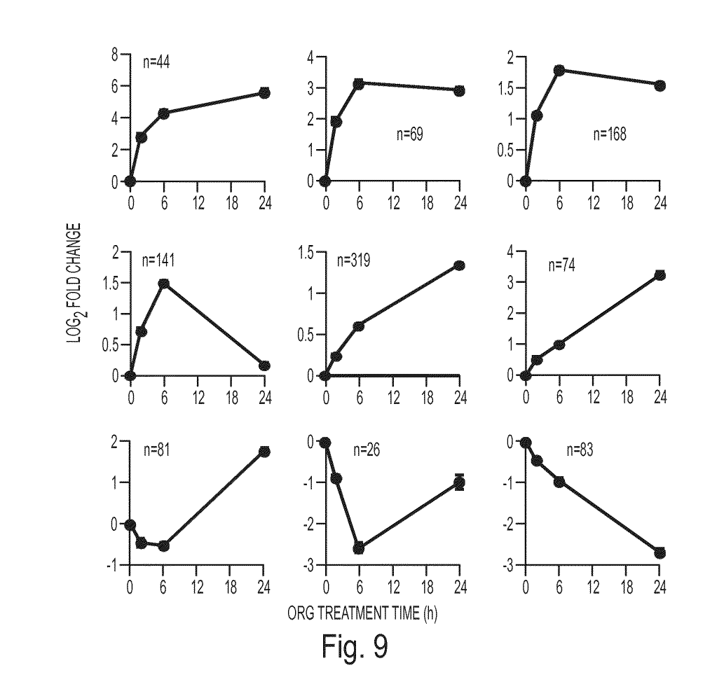

Self-organizing map (SOM) clustering of progestin-regulated transcripts associated with PR binding regions in T-47D cells (FIG. 2E) and analysis of corresponding binding regions showed that PR binding regions were significantly closer to the TSSs of rapidly up-regulated genes, than to TSSs of down-regulated genes, or genes regulated at a later time point (Kruskal-Wallis one-way analysis of variance, p value <0.001, FIG. 2F—compare clusters 0-5 with 6-8). Self-organizing map clustering of all progestin regulated transcripts revealed a pattern of regulation that was overall similar to that observed with the subset of genes associated with PR binding (FIG. 9). However, a cluster of 26 transcripts was detected in the larger dataset representing transcripts that were decreased at all time points by ORG2058, but began to return toward basal levels at 24 h. Although 12 transcripts in this cluster were also present in the set of transcripts associated with PR binding, 14 were found only in the full progestin regulated transcriptome, and represented transcripts that were regulated early but largely recovered by 24 h, suggesting that transcriptional silencing mediated by direct PR binding may be more sustained than indirect regulation.

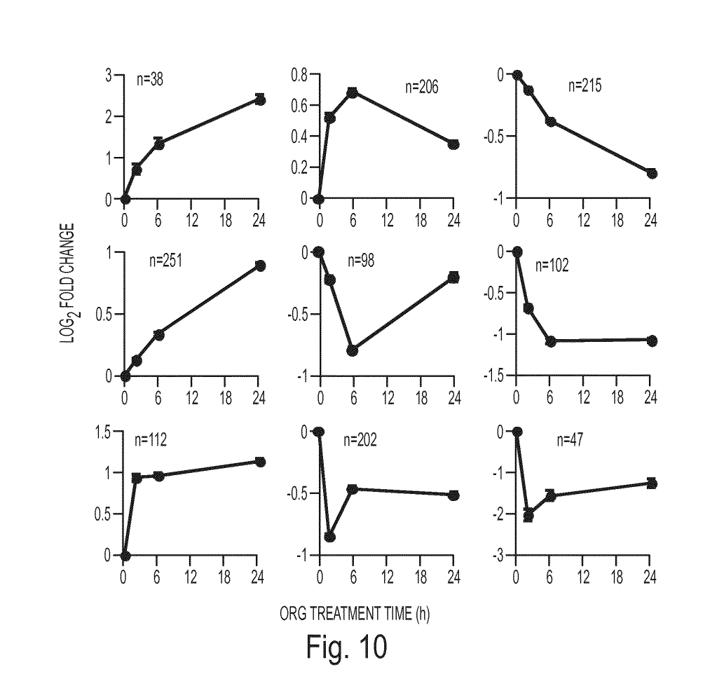

In AB32 cells, more transcripts were decreased than increased by ORG2058 (FIG. 10) and there was no significant difference in mean binding region to TSS distance observed between regulation clusters (Kruskal-Wallis p value=0.209, not shown).

### Example 4

### Distinct Patterns of PR Binding Between Cell Lines Reflects Divergent Transcriptional Response

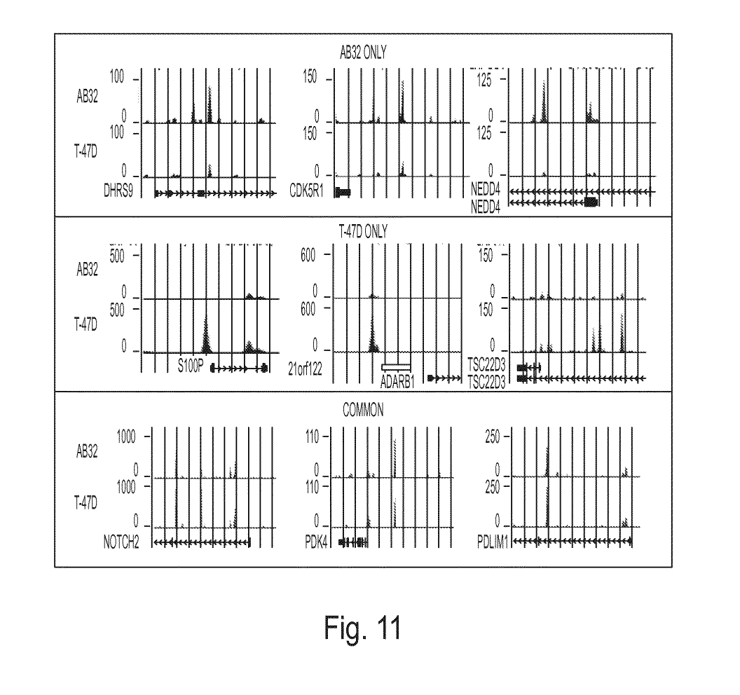

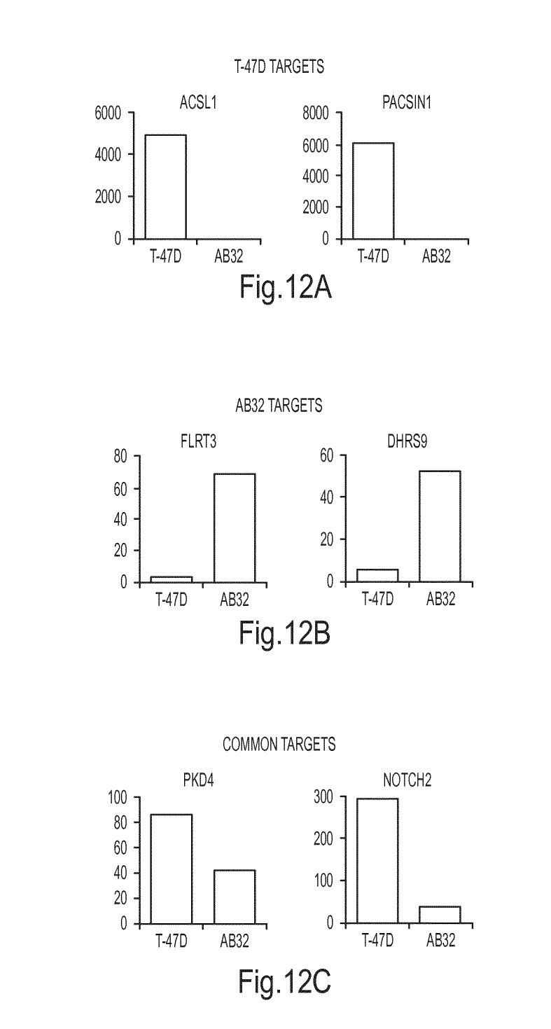

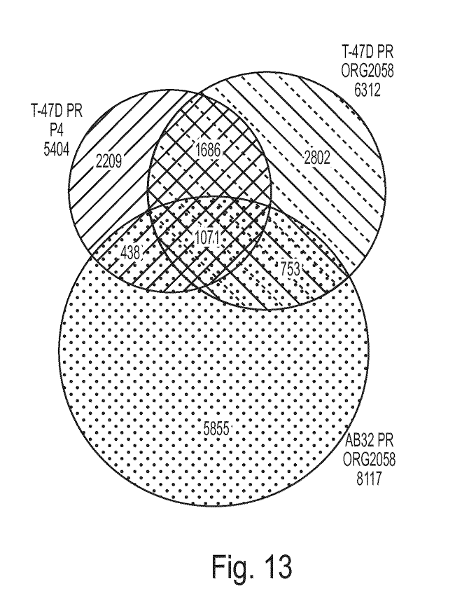

Of the 6312 regions bound by PR in T-47D and 8117 in AB32, just 1824 binding regions (14% of the combined total) were common to both cell lines, representing 29% of binding regions in T-47D and 22% of AB32 binding regions (FIG. 3). The binding regions common to both cell lines were not more likely to be associated with regulated genes: of the 1824 binding regions found in both AB32 and T-47D, 431 (24%) were associated with progestin regulation in AB32 and 345 (19%) in T-47D—similar to the association of all binding regions with regulated genes shown in Table 1. 157 (9%) binding regions were associated with regulated genes in both cell lines (data not shown). Examples of binding peaks detected exclusively in one cell line or common to both are shown in FIG. 11. Directed ChIP confirmed the differential patterns of PR binding to genes regulated in AB32, T-47D or both cell lines (FIG. 12). Moreover, direct examination of the overlap between PR binding in T-47D and AB32 cells with another PR cistrome in T-47D cells [40] revealed a higher overlap in binding regions between the two T-47D data sets than to the AB32 PR data set (FIG. 13).

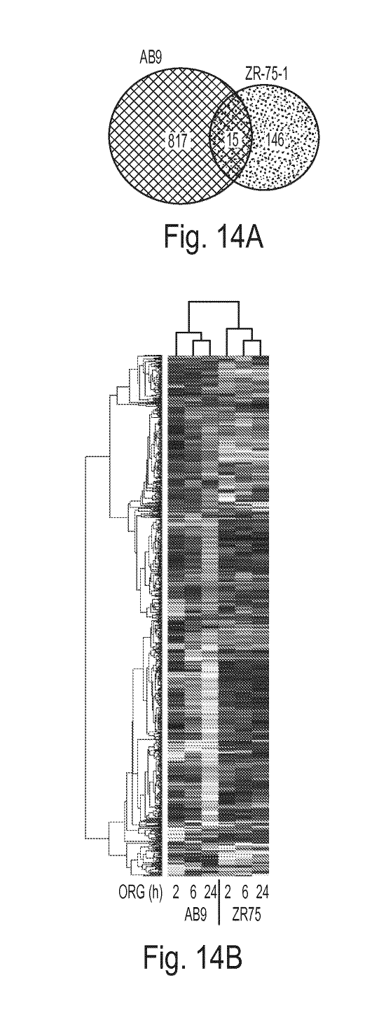

The lack of overlap in binding sites between the two cell lines was reflected in a similarly low overlap in transcriptional profiles at 2, 6 and 24 h of progestin treatment (FIG. 3C-E). The small overlap in progestin targets in the two cell lines was similar at all time points examined (FIG. 3E). This lack of overlap was confirmed in two additional cell lines, ZR-75-1 breast cancer cells and an additional PR expressing cell line MCF-10A clone, AB9, which revealed a similarly low overlap of progestin response when compared directly with each other (FIG. 14) or with the T-47D or AB32 cells.

### Example 5

### Conserved PRE Identified in PR Binding Regions in T-47D and AB32 Cells

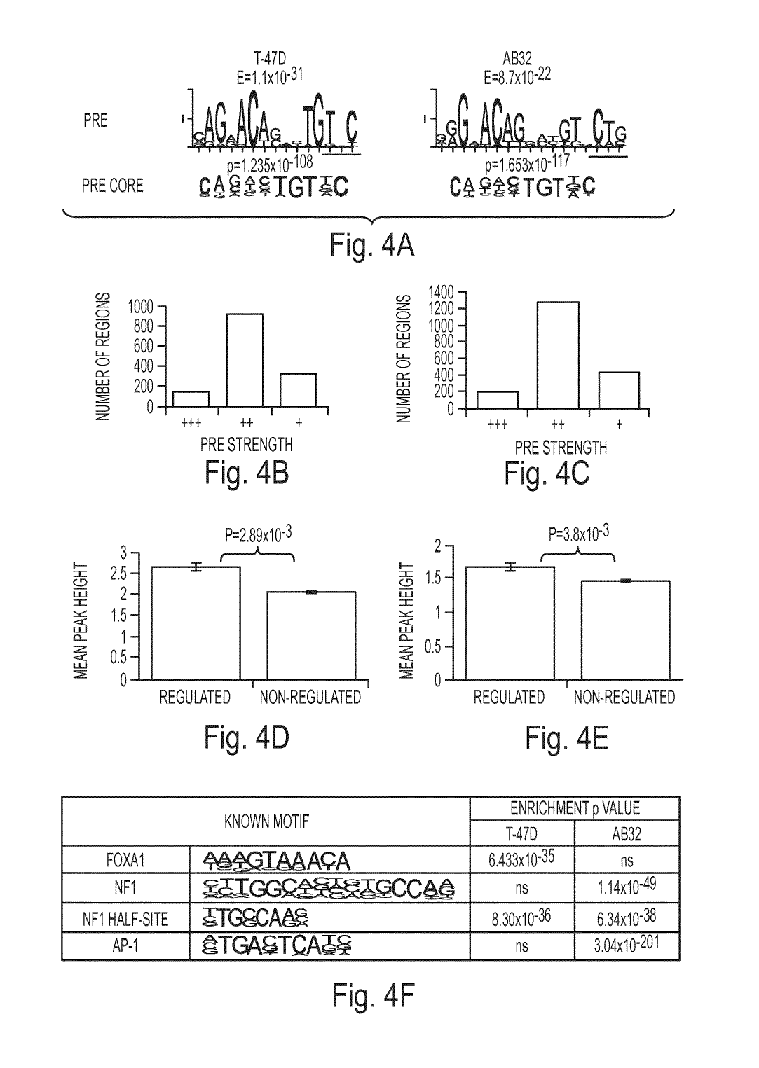

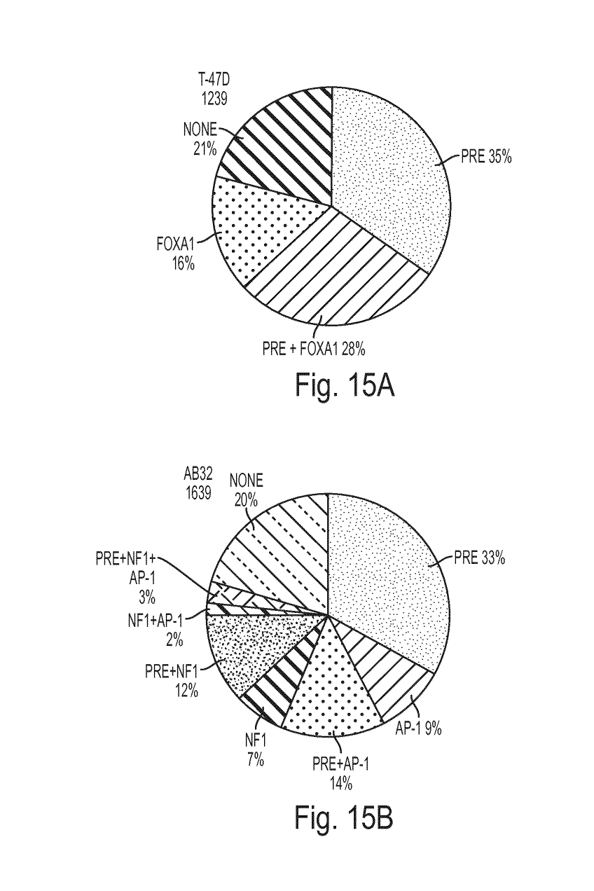

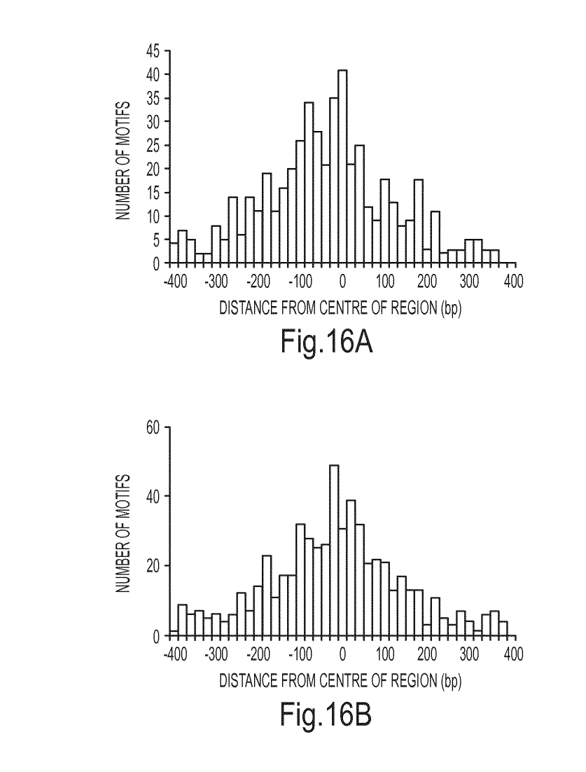

De novo motif enrichment analysis of PR binding regions associated with regulated genes identified highly significant enrichment of a conserved PR binding motif (FIG. 4A) consistent with previously predicted progesterone response elements (PREs). Both the MEME-ChIP (software analytic program) and Homer motif analysis (software analytic program) tools identified a consensus PR binding motif consisting of a six base pair inverted repeat sequence RGNACA separated by three non-specific bases, in agreement with classical biochemical studies of PR binding elements [2, 3] and similar to the element briefly described recently [40]. In addition, de novo analysis in Homer identified a highly enriched shorter element, representing the central core of the inverted repeat sequence (FIG. 4A), suggesting that this more strongly conserved part of the PRE is most critical for PR binding. In T-47D cells 782/1239 PR binding regions (63%) associated with regulated genes contained one or more full-length or core PRE motifs (FIG. 15A) and in 33% of binding regions this included at least one highly conserved full-length PRE. In AB32 cells 62% of regulation-associated PR binding regions contained PREs (FIG. 15B). A substantial proportion of PR binding was likely to be mediated by a direct genomic interaction with these motifs, as there was a normal distribution of PREs about the centre of binding regions in both T-47D and AB32 cells (FIG. 16, and one sample Kolmogorov-Smirnov test). A number of binding regions in both cell lines contained more than one PRE, although number of PREs was not correlated with peak height (Pearson regression R-squared=0.0048), suggesting that PRE number was not correlated with binding strength.

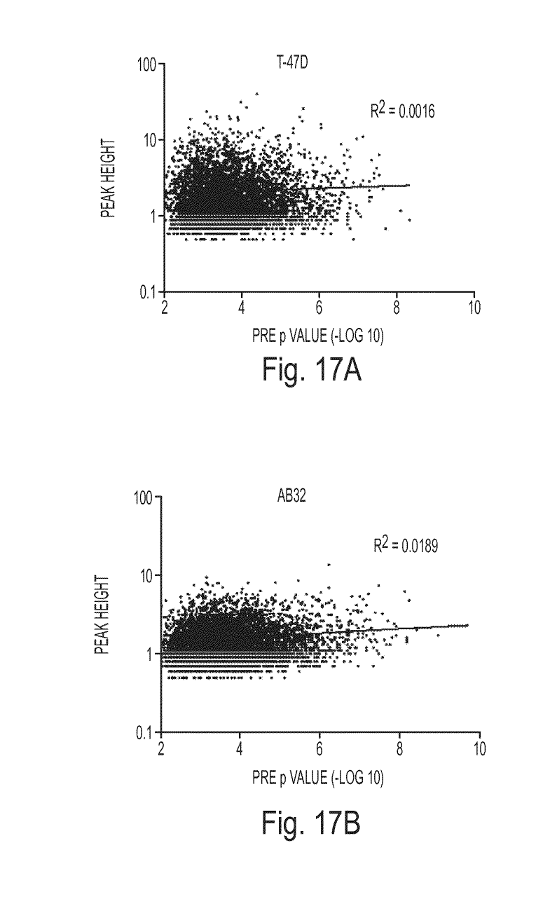

The position specific probability matrix for the full-length PREs defined by de novo motif mapping in the two cell lines was used to classify PRE strength in all PR binding regions. PRE strength did not predict a transcriptional outcome, since the same proportion of regulation associated and non-associated PR binding regions contained strong PREs (data not shown). Regulation-associated PR binding regions were grouped based on PRE p value (FIG. 4B-C, strong (+++)—p<1×10⁻⁵, moderate (++)—p=1×10-5-1×10-3, weak/absent (+)—p>1×10⁻³). By these criteria 77% regulation-associated binding regions in T-47D cells and 76% in AB32 contained one or more moderate or strong PRE (p<0.001). The majority of PR binding regions did not contain a strong PRE, suggesting broad flexibility in PR binding site selection and also implying that PR binding strength is not just determined by the DNA nucleic acid sequence and is likely influenced by secondary DNA structure and other DNA binding PR cofactors. PR binding peak height was positively correlated with transcriptional outcome, suggesting that it is a measure of binding strength. In both T-47D and AB32 cells, the average peak height of binding regions that were within 50 kb of an up-regulated gene was significantly greater than those that were distant from any regulated gene (FIG. 4D-E, unpaired t test, T-47D p=2.89×10-8, AB32 p=3.8×10-6). When PRE strength was compared directly with peak height, no correlation was observed, demonstrating that PRE quality alone does not determine PR binding strength (FIG. 17). This finding was supported by directed ChIP validation of the top PR binding regions by peak height in T-47D and AB32 cells. Analysis of these regions revealed just two regions containing strong PREs in T-47D and three regions in AB32 cells. Most regions in the top ten contained moderate strength PREs, and binding of PR was confirmed in all but one binding region in each cell line (Table S2).

### Example 6

### Binding Regions in T-47D and AB32 have Distinct Motif Enrichment

We analyzed the sequences up to 400 bp from each binding peak for the presence of other enriched motifs. Binding regions in T-47D cells were significantly enriched with motifs for the pioneer factor FOXA1 (FIG. 4F), whereas there was no significant enrichment for this factor in PR binding regions from AB32 cells. FOXA1 binding motifs were identified in 548/1239 (44%) of regulation-associated PR binding regions in T-47D cells (FIG. 15A). In binding regions in AB32 cells there was strong enrichment of binding sites for the AP-1 complex and for the DNA binding PR cofactor NF1. AP-1 binding motifs were present in 454/1639 (28%) and NF1 sites were identified in 380/1639 (23%) of regulation-associated PR binding regions in AB32 cells (FIG. 15B). Relatively few binding regions in these cells (74/1639, 4.5%) contained binding motifs for both factors. There was no difference in the prevalence of any of the transcription factor motifs in binding regions near genes that were up or down-regulated by ORG2058 (t test, p>0.05). Moreover, separate motif analysis of up-regulation associated binding regions and of those associated with down-regulation did not reveal enrichment of different transcription factor motifs (not shown).

### Example 7

### The Pioneer Factor FOXA1 Alters PR Transcriptional Response

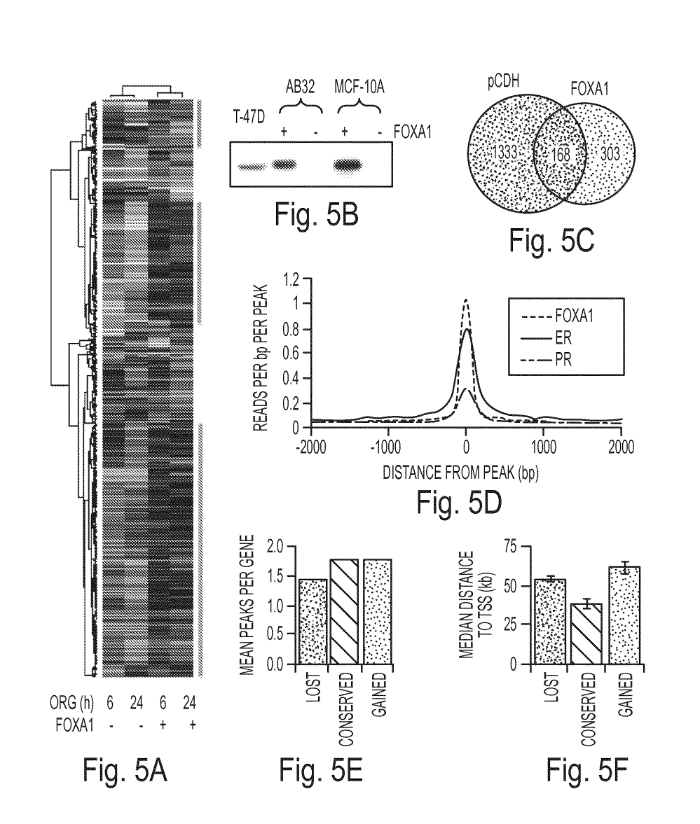

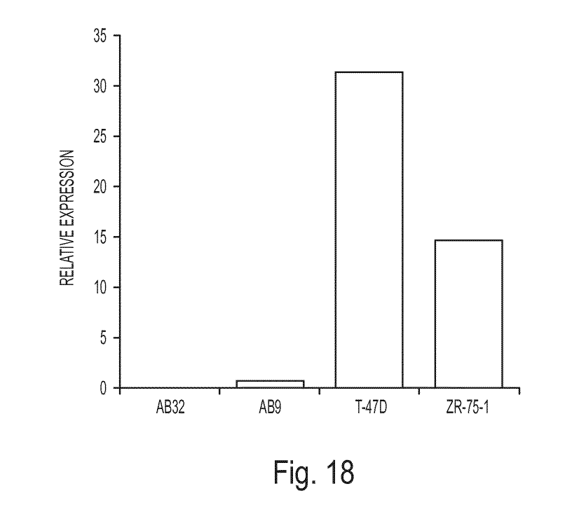

FOXA1 transcripts were abundantly expressed in T-47D and ZR-75-1 cells relative to AB32 and AB9 cells (FIG. 18), suggesting that endogenous levels of FOXA1 may play a role in regulating the PR transcriptional response. Accordingly, AB32 cells lacking endogenous levels of FOXA1 were infected with lentiviral-delivered FOXA1 (FIG. 5). This resulted in a profound alteration in the progestin-regulated transcriptome at 6 h and 24 h (FIG. 5A). FOXA1 transduction resulted in progestin regulation of 303 transcripts that were not regulated in cells transduced only with the control pCDH virus (FIG. 5C). Almost half of these targets (146/303, 48%) were detected as a distinct cassette of genes that clustered together (FIG. 5A, red bar). Functional analysis revealed that genes that gained progestin regulation after FOXA1 expression were involved in blood vessel morphogenesis and regulation of cell motility (Table S3 and data not shown). These categories included genes such as transforming growth factor β3, CD44 and basic fibroblast growth factor, suggesting a broader developmental function. Surprisingly, a large proportion of transcripts that were regulated when FOXA1 was not present (1333 transcripts regulated at 6 h, 24 h or both, in absence of FOXA1, FIG. 5C), lost regulation upon expression of the pioneer factor and were evident in multiple clusters (FIG. 5A, blue bars). Functional analysis revealed a major impact of FOXA1 expression on genes involved in negative regulation of apoptosis: these had been increased by progestins in absence of FOXA1, but lost progestin responsiveness when FOXA1 was expressed (Table S3 and data not shown). Genes in this category that were decreased by progestin were unchanged by FOXA1 expression, suggesting that the net effect of FOXA1 was to promote apoptosis in response to progestin. The dampening effect of FOXA1 expression on progestin regulation suggested that the pioneer factor may play a dual role in PR action, similar to its role in androgen receptor signaling where it acts as an activator on a subset of androgen targets and a co-repressor on others [41]. The progestin regulation of just 168 transcripts was unaffected by changed FOXA1 levels (FIG. 5C). Functional analysis of these genes revealed that progestin-mediated increases in genes involved in cell cycle progression, suggesting that the proliferative effects of progestin may not require FOXA 1.

As FOXA1 appeared to have an effect on PR transcription distinct from that observed for ER, we compared the density of FOXA1 ChIP-seq interactions [42] around PR binding regions in T-47D cells, with those observed at FOXA1 or ER binding regions (FIG. 5D). Without being bound by theory, binding of FOXA1 around ER binding regions was very high, indicating a requirement for this factor in estrogen signaling. In contrast, although a peak of FOXA1 interaction was seen near PR binding regions, sequence enrichment was significantly lower (FIG. 5D) suggesting that while FOXA1 may be involved in PR binding at some regions, it represents a subset of binding events.

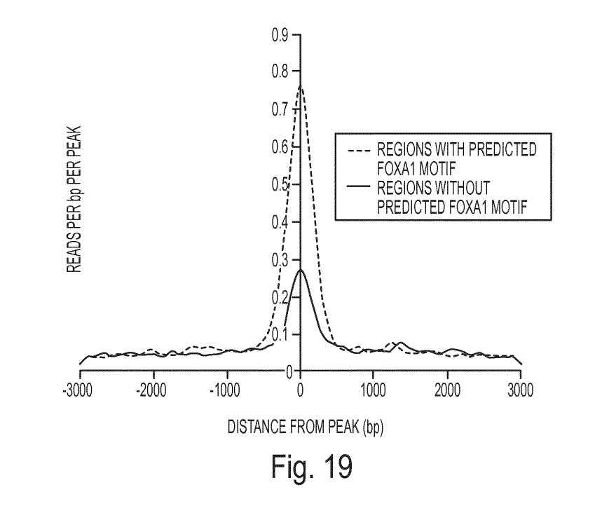

This conclusion is supported by the finding that FOXA1 binding was much stronger at PR binding regions in which a FOXA1 motif had been predicted, than in regions where no motif was found, and was similar to the density of binding observed overall in ER binding regions (FIG. 19). In order to test whether PR binding site numbers were different near genes that gained progestin-regulation upon FOXA1 expression, we compared the number of PR binding peaks in FOXA1 negative AB32 cells that were near to genes that lost, gained or retained progestin regulation when FOXA1 was expressed. Although there were slightly fewer PR binding regions near genes that gained regulation (FIG. 5E), the difference was not significant. This suggested that the capacity of FOXA1 to influence PR binding and transcriptional regulation of target genes may not relate to PR binding site density; PR may form weak associations near to the “gained” subset of genes, but FOXA1 was required for the interaction to become productive. We also examined the level of enrichment of motifs for NF1 and AP-1 in PR binding regions associated with genes that lost, gained or conserved progestin regulation when FOXA1 was expressed and found no difference between the groups (not shown).

FOXA1 influences transcription factor activity via its DNA bending activity [43, 44, 45]. We speculated that PR binding regions that require FOXA1 to affect transcription may be further from the target gene than those that do not, and that binding of FOXA1 near those regions results in DNA bending, which brings the PR transcriptional complex closer to the target gene. Examination of the distance from PR binding regions to genes that gained regulation by FOXA1 revealed that this was the case and that this subset of regions was significantly further from the regulated gene than binding regions near genes regulated in the absence of FOXA1 (FIG. 5F, p=0.003, unpaired t test).

In summary, ChIP-seq profiling in two different exemplary cell lines (e.g., T-47D and AB32) has revealed unexpectedly distinct patterns of PR binding. These distinct cistromes are reflected in marked differences in transcriptional response to progestins. PR binding in the two cell lines is mediated by highly similar PREs, demonstrating a similar mode of DNA interaction, but key differences in cofactor binding site enrichment, particularly FOXA1, suggest that the expression levels of these cofactors influence cell-specific binding and ligand response of PR.

This first detailed genome-wide survey of PR genomic interaction has identified non-overlapping PR binding sites in immortalized normal and malignant breast cells; shown that PR interactions occurred distal to proximal promoters, supporting the view that PR effects are mediated over a longer distance than has previously been expected for direct cis-acting transcription factors; and demonstrated that transcriptional cofactors are important contributors to cell-specific PR activity.

### Example 8

### PR Binding Regions are Distant from TSS

Most PR binding regions were located more than 10 kb from the TSS of regulated genes, with less than 35% of regulated genes in both cell lines having PR binding regions within 10 kb of the TSS, and less than 4% of regulated genes having binding regions within 1 kb of the TSS. In both breast cell types, binding was correlated with gene regulation, with most progestin-regulated genes having one or more PR binding regions within 50 kb, and genes increased by progestin being more likely to be associated with PR binding sites than genes that were decreased. These findings are consistent with observations for other nuclear receptors in comparable experimental systems. Reddy et al identified 4392 GR binding sites (2% FDR) by ChIP-seq in dexamethasone-treated A549 cells [46]. Welboren et al identified between 7713 and 10205 estrogen-dependent ER binding sites, depending on the peak-calling algorithm used, in MCF-7 cells [47]. Both ER and GR demonstrate a correlation between binding and gene regulation, and in line with the findings of this study, a relatively low proportion of promoter proximal binding is reported [14, 46, 47]. The stronger correlation between binding and transcriptional up-regulation than down-regulation has also been described for ER [14] and GR [46].

In one aspect, the number of PR binding sites discovered exceeded the number of progestin-regulated transcriptional targets and many PR binding sites were not associated with active transcription, (e.g., 20% of PR binding regions were associated with transcriptional regulation in each cell line). In one aspect, this is thought to represent the associated “closeness” of PR binding sites to the genes. This finding is consistent with results for other nuclear receptors [14, 17, 46, 47]. A number of potential explanations are proposed [48]. Some binding events may regulate transcription at a level below the detection threshold of genome-wide expression profiling. Moreover, a subset of binding sites may represent weaker associations or binding occurring in only a subset of cells such that transcriptional regulation does not occur at a significant level. Our data support this possibility, since PR binding peak signal strength was significantly higher near regulated genes compared to non-regulation associated binding regions. It has also been suggested that binding events that are not associated with transcriptional regulation may be at cell type specific sites requiring the co-operation of binding cofactors that are available only in a subset of contexts [48, 49, 50]. It must also be assumed that a proportion of binding regions represent non-specific interactions, although the finding that PREs are similarly prevalent in regulation-associated and non-associated binding regions would argue that non-specific interaction explains a small component of overall binding.

### Example 9

### PREs in PR Binding Regions

PR binding regions were significantly enriched for a binding element with a sequence consistent with previously described progesterone response elements [2, 3]. The relative conservation at specific base positions in the 15 base pair palindromic response elements was variable, and was consistent with the pattern of conservation seen for GR [46, 50] and AR [51]. A shorter motif, representing the core highly conserved elements (CA/t nnn TGTnC, FIG. 4A), was also detected, demonstrating the particular importance of these positions in the PR binding element. There was a high degree of variability of PRE sequences, as indicated by the consensus sequence allowing for marked variation at several positions, and many binding regions contained weak sequences that diverged considerably from the ideal. Moreover, a proportion of PR binding regions totally lacked a consensus PRE, raising the question of whether these were directly binding PR. To address this, we sought motif enrichment just in those regions, and did identify a PRE-like motif at a lower level of significance (not shown). This suggests that many binding regions lacking consensus PREs do contain sequences consistent with a PRE. A similar finding was reported for GR [46].

Although there was variability in the presence and strength of PREs identified in PR binding regions, this was not a determinant of whether a particular region was associated with transcriptional activity, as PRE strength was not correlated either with PR binding peak strength or with transcriptional outcome. This suggests that PRE strength is not the sole determinant of whether PR will interact with a particular binding region and regulate gene expression, and that other sequence features and the influence of DNA binding cofactors are likely to be important determinants. This is supported by the identification of FOXA1, AP-1 and NF1 as potential cell type-specific binding cofactors for PR in the two cell lines examined.

### Example 10

### The PR Cistromes in T-47D and AB32 Cells are Non-Overlapping

There was a relatively small overlap in PR binding regions in T-47D and AB32 cells. This was consistent with the observation that the transcriptional response to progestin was also non-overlapping between the two cell lines. Moreover, binding regions that were common to both cell lines were not more likely to be associated with a transcriptional outcome. Expression profiling in two additional cell lines, ZR-75-1 breast cancer cells and an independent PR positive MCF-10A clone (AB9), revealed a similarly low overlap in transcriptional regulation by progestins. Comparison of ER binding patterns in MCF-7 breast cancer cells and ER expressing U20S osteosarcoma cells revealed a similarly low overlap in binding sites and transcriptomes [49]. In that study, differential promoter methylation was proposed to underlie this difference. However, global inhibition of DNA methylation in AB32 cells, while enhancing existing transcriptional targets, did not significantly alter the progestin-responsive transcriptome (data not shown).

In support of our findings, Yin and colleagues have recently reported a similarly low overlap in PR genomic interactions in T-47D cells and uterine leiomyoma cells on exposure to the antagonist RU486 [52]. Comparison of the exemplary T-47D and AB32 PR cistromes described herein with a publicly available dataset revealed a greater overlap between the two T-47D datasets (51% reported T-47D PR binding sites were also found in T-47D in our study) than with binding in AB32 cells (28%), supporting the validity of the observation of distinct binding patterns.

In the published study, T-47D cells were treated with progesterone, which has a similar pharmacokinetic profile to ORG2058, but dissociates from PR more rapidly than the synthetic analogue. Moreover, the mobility of PR at genomic DNA has been shown to be ligand specific [53] and may differ when bound to ORG2058 compared to progesterone. Secondly, PR binding was detected by different methods: in this study ChIP-seq was used, whereas the published data are derived from ChIP-chip. ChIP-seq surveys binding in an unbiased genome-wide fashion. ChIP-chip is dependent on the sequences present on the arrays used and can be affected by hybridization bias. A similar overlap was observed in ER binding sites detected in MCF-7 cells by ChIP-seq and ChIP-chip [47]. Lastly, the analysis methods used to generate the published data were different than used in our study.

### Example 11

### Role of Chromatin Structure and the Pioneer Factor FOXA1

Pioneer factors such as FOXA1 are able to bind to tightly packed heterochromatin, opening DNA structure to allow binding and regulation by nuclear receptors, including ER, GR and AR [12, 14, 15, 17, 41, 54]. The level of requirement for FOXA1 and the role that it plays in receptor signaling differs between the receptors. Expression of FOXA1 is critical for transcriptional activation by ER, although the specific gene targets may differ between cell lines. In a recent study, Hurtado and colleagues mapped ER and FOXA1 binding in three breast cancer cell lines, MCF-7, T-47D and ZR-75-1, and determined that positioning of the silencing factor CTCF was different between the three cell lines and defined which ER targets were transcriptionally enhanced by FOXA1 binding. In these cell lines FOXA1 was critical for ER action [15].

In contrast, FOXA1 appears to play a dual role in androgen signalling, where it promotes androgen responsiveness of some AR targets and acts as a repressor of others. This is supported by a recent study in LNCaP prostate cancer cells where depletion of FOXA1 caused significant remodeling of AR binding patterns and a marked increase in androgen regulated transcripts [41]. In this context FOXA1 is a determinant of binding site selection and acts both as a facilitator and a repressor of AR binding depending on the target site.

Our data suggest that FOXA1 may play a similar role in PR signaling as with AR, since FOXA1 was not absolutely required for progestin response and over-expression of FOXA1 in AB32 cells, which lacked endogenous FOXA1, caused a marked decrease in the number of progestin-regulated genes in those cells. In T-47D cells where FOXA1 is abundantly expressed, binding motifs for the pioneer factor were statistically enriched in PR binding regions. The role of FOXA1 in PR signaling through regions containing FOXA1 motifs was supported by the finding that FOXA1 binding levels at these sites in T-47D cells was greater than interactions at PR binding regions that did not contain a predicted FOXA1 motif. However, a comparison of average FOXA1 binding around all PR binding regions in T-47D cells with those at ER interaction sites revealed significantly lower overall enrichment of FOXA1 binding near PR than ER, suggesting that FOXA1 is not required for all PR interactions. Taken together, our data suggest that FOXA1 may act as an enhancer of PR transcriptional activation of many of the targets identified in T-47D, whereas in AB32 cells the lack of FOXA1 expression allows binding of PR targets that may normally be repressed by FOXA1.

The overlap between progestin regulation in T-47D and FOXA1 transduced AB32 cells was low, suggesting that FOXA1 expression did not cause AB32 cells to become more like T-47D cells in their progestin response. This is consistent with our observation that FOXA1 may not be absolutely required for all PR binding events in T-47D cells. It also suggests that, although FOXA1 may affect PR binding, other cell specific factors or characteristics are important in determining PR binding, which may not be identifiable by ChIP. Both ER and AR have been shown to associate with histone modifying factors in a cell-type and promoter-specific fashion [55, 56], which are recruited to enhancers as part of a large co-regulatory complex and would not be identifiable through motif analysis. The nature of the GR cistrome has been shown to be highly dependent on chromatin accessibility [57], which is also cell type specific. It is likely that the same factors influence PR binding in a cell type specific fashion.

### Example 12

### AP-1 and NF-1

Nuclear receptors, including PR, have been shown to associate with DNA independently of hormone response elements, by tethering to AP-1 [18,19, 20]. In the case of ER, this mechanism was reported to mediate transcriptional repression of target transcripts by estrogen [14]. These findings suggest that AP-1 binding sites may be more common in binding regions that lack PREs and could be associated with down-regulated genes. AP-1 sites were present in 27% of regions that contained PREs and 29% of regions lacking PREs in AB32 binding regions. This proportion was higher overall than in T-47D cells where AP-1 site enrichment was not observed (12% regions with PREs and 10.7% regions lacking PREs contained AP-1 sites in T-47D), however it was not different between the two subsets of binding regions. There was also no evidence that AP1 sites were more prevalent in down-regulated than up-regulated genes (data not shown). These data suggest that, while AP-1 may cooperate with PR on a subset of binding sites in AB32 cells, its role in progesterone signaling may be more minor than for estrogen.

Binding of the transcriptional cofactor NF1 to DNA requires co-association by PR, and NF1 and PR have synergistic effects on gene expression [11], demonstrating the potential for co-expression of these transcription factors to result in enhanced transcriptional outcomes. In the mammary gland, the coordinated expression of NF1 isoforms is involved in controlling lactation and involution [58]. NF1 action in the mammary gland is context-specific, and is induced when mammary epithelial cells are maintained in contact with laminin-rich extracellular matrix [59]. The development-specific and context-specific actions of NF1 in the mammary gland suggest that its interplay with PR may be regulated by both NF1 and PR levels, and that these may be susceptible to modulation under physiological circumstances that include carcinogenesis. Enrichment of NF1 binding motifs in PR binding regions in AB32 cells, but not breast cancer cells, supports this view and suggests that NF1 is a cell type-restricted PR cofactor.

The combination of chromatin remodelling cofactors is important for progesterone response in the breast and that the relative expression and coordinated action of these cofactors determines the PR cistrome. Progesterone has a diverse range of effects in normal and malignant target tissues and the results of this study demonstrate that the interplay between key cofactors and PR on the progesterone regulated cistrome contributes to context specificity of progesterone action, and plays a central role in aberrant progestin effects in breast cancer.

### Example 13

### Cell Culture

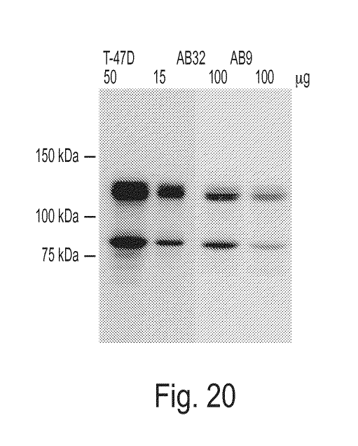

T-47D and ZR-75-1 breast cancer cell lines were obtained from the E.G. and G. Mason Research Institute (Worcester, Mass.). MCF-10A immortalized normal breast cells and HEK293T kidney cells were obtained from the American Type Culture Collection (atcc.org, Manassas, Va.). T-47D and ZR-75-1 cells were maintained in RPMI1640 medium containing 10% fetal calf serum and 0.25 units/ml insulin. HEK293T were maintained in Dulbecco's Modified Eagle's Medium, supplemented with 10% fetal calf serum. The AB32 and AB9 cell lines were generated by co-introduction of PRA and PRB from viral vectors into the MCF-10A cell line and clonal selection using puromycin. Clones were characterized by dual immunofluorescent analysis and by western blotting for expression of PRA and PRB. A western blot comparing PR expression in AB32 and AB9 with PR levels in T-47D cells is shown in FIG. 20. The cells were maintained in 1:1 DMEM:Hams-F12 medium supplemented with cholera toxin (0.1 μg/ml), insulin (0.28 iu/ml), hydrocortisone (0.5 μg/ml), epidermal growth factor (0.02 μg/μl), and 5% horse serum. The synthetic progestin ORG2058 was obtained from Amersham Biosciences (GE Healthcare, Rydalmere, Australia). TSA and 5AdC were obtained from Sigma-Aldrich (Castle Hill, Australia).

### Example 14

### Chromatin Immunoprecipitation

Cells were cultured to 80% confluency in 15 cm tissue culture dishes, then treated for 45 minutes with 10 nM ORG2058 or vehicle. Chromatin was subsequently cross-linked by the addition of formaldehyde to the culture medium to a final concentration of 1% and incubation for 10 minutes at 37° C. Media were immediately removed and the cells were washed with cold phosphate buffered saline and harvested by scraping. Cells were collected by centrifugation and pellets were lysed 10 minutes at 4° C. in SDS buffer (1% SDS; 10 mM EDTA; 50 mM Tris-HCl, pH 8). The lysates were sonicated at 4° C. with a Branson 450 sonicator, using seven one minute bursts at 40% amplitude and 60% duty, each separated by a rest of at least one minute. Lysates were centrifuged at 13,000×g at 4° C. for 15 minutes to remove debris. Genomic DNA was isolated from an aliquot of lysate and checked by gel electrophoresis to confirm that sonication had resulted in fragmented DNA with an average size of 200 to 400 bp. Supernatants were diluted 1:10 with IP buffer (0.5% NP-40; 50 mM Tris, pH 8; 120 mM NaCl; 0.5 mM PMSF; Complete protease inhibitor cocktail, Roche, Ryde, Australia) and pre-cleared by incubation with Dynabeads M-280 sheep anti-mouse IgG magnetic beads (Invitrogen, Mulgrave, Australia), with gentle rotation at 4° C. for at least 2 h to reduce non-specific binding to the secondary antibody beads. Genomic DNA fragments that were bound to PR were isolated by rotation overnight at 4° C. with in-house hPRa6 and hPRa7 mouse monoclonal anti-PR antibodies [60] and fresh sheep anti-mouse IgG magnetic beads (40 ul per 1.4 ml diluted lysate). Beads were washed sequentially with IP buffer, high salt wash buffer (0.5% NP-40, 50 mM Tris, pH 8, 500 mM NaCl, 0.5 mM PMSF), lithium wash buffer (250 mM LiCl, 0.5% NP-40, 1% sodium deoxycholate, 1 mM EDTA, pH 8, 10 mM Tris-HCl, pH 8) and TE (10 mM Tris, pH 8, 1 mM EDTA). Isolated DNA fragments were eluted twice for 15 minutes at room temperature using elution buffer (1% SDS, 0.1M NaHCO3). Cross-links were reversed by addition of 0.25M NaCl and heating to 65° C. for at least 6 h. DNA fragments were purified using Qiagen PCR purification columns (Qiagen, Doncaster, Australia). DNA fragments isolated by PR chromatin immunoprecipitation from ORG2058 treated cells were sequenced on an Illumina GA-IIx sequencer at the Ramaciotti Centre for Gene Function Analysis (University of New South Wales, Australia) and GeneWorks (Hindmarsh, Australia). Input DNA isolated from the pre-cleared ORG2058 treated samples were sequenced as a baseline control. An aliquot of DNA was isolated from the ORG2058-treated, pre-cleared sample, to serve as an input DNA control sample in next-generation sequencing.

### Example 15

### Analysis

Sequences were aligned to repeat masked human genome hg18 (NCBI build 36) using Bowtie 0.12.0.1 [37]. Up to 2 mismatches were allowed in the aligned sequences. Multiple alignments were permitted up to a multiplicity of 10, but only the best ranked alignment was reported. This strategy resulted in alignment of 42% to 48% of reads. Results in T-47D represent the combined outcome of three independent biological replicates and two replicate input controls. AB32 results are from two independent ChIP and input control samples each. All sequences were at 36 bp read length except for one ChIP and one matched input control sample from T-47D cells. These samples were processed with a 64 base pair read length, but were trimmed to 36 base pairs during data processing to avoid bias in the downstream analysis. Enriched regions of PR binding were determined using the ERANGE open source software tool [38]. Peak shift was determined to be 70 bp using the -shift Learn function in ERANGE findall.py (analytical software program). The peak threshold was set at four-fold background as determined from the control input DNA sequence. The minimum number of reads (RPM) within a region was set to 10. Multireads were weighted at a value of 1/multiplicity. Peaks were called with false discovery rate 0.27%. Regions of PR binding were annotated with respect to neighboring genes using CisGenome v1.1 [61] and Homer [62]. Known and de novo enriched binding motifs were identified using Homer and the MEME suite of motif analysis tools, version 4 [63, 64]. Significance of enrichment of binding motifs discovered in MEME was determined using a Fisher Exact Test. The E-value for enrichment represents the p-value multiplied by the number of sequences tested. Motif enrichment was scored in Homer using a cumulative hypergeometric distribution analysis comparing binding region sequences with a matched genomic background [62]. The FIMO program in MEME was used to classify full-length PRE occurrences in PR binding regions in AB32 and T-47D cells, using the position specific probability matrices discovered by MEME in the two cell lines. Sequences with a p value <0.01 for similarity to the consensus PRE were reported and p values ranged from 0.01 to 1×10-10. A lower p value signified greater sequence conservation compared to the consensus PRE and for the purposes of comparisons, a p value <1×10-5 was considered to represent a strong PRE. For comparison of PR genomic interaction in T-47D cells with published ER and FOXA1 interactomes [42], sequence tag libraries were generated from all three data sets in Homer and binding peaks were identified using the same parameters for each data set. Average FOXA1 tag density was then determined at PR, ER and FOXA1 peaks using the peak annotation function in Homer. All raw data generated by ChIP-seq and gene expression profiling have been deposited on the Gene Expression Omnibus (www.ncbi.nlm.nih.gov/geo/) and can be accessed through GEO accession number GSE31130. Gene expression data conform to MIAME guidelines.

### Example 16

### Real-Time PCR

Directed ChIP was performed using the same protocol as described for ChIP-seq. Target templates were quantitated using Platinum Sybr Green reagents (Invitrogen) in a RotorGene 6000 real-time PCR machine. Directed ChIP was carried out as described above and purified gDNA fragments were diluted 2 to 5-fold prior to quantitation by real-time qPCR. Primer sequences used for specific target validation were: ACSL1-fwd 5′-TGC AAA GAG CAA GAC AGA AAA G-3′, rev-5′-GCG GTC ATA GAG ACA CAA TTC C-3′, DHRS9-fwd 5′-GGC TGT CTG AGT GAA TCT GTA GTG-3′, rev-5′-AGT TAC ATT TGC CCT TGA TTC C-3′, FLRT3-fwd 5′-GGA GAA ACA GAC TTT ACC TGA CC-3′, rev-5′-TGT TGC AGT CAA GGA GAC AGA G-3′, NOTCH 2-fwd 5′-GCC TGT TCC TAT TAA GTG TCC TG-3′, rev-5′-GGC TGT AAA GTT ATT TGC TAG ATT G-3′, PACSIN1-fwd 5′-AAC GTC CTC TTC CTG CTC TTG-3′, rev-5′-GAG CTT TGA TGT AGA CGG AAT-3′ G, PDK4-fwd 5′-CG AGC AGC AAT AAC TTT CC-3′, rev-5′-ACG CAA GAA CAC AGT GAG TAG C-3′.

### Example 17

### Lentiviral Transduction

The FOXA1 cDNA was obtained from the PlasmID Dana Faber/Harvard Cancer Center DNA Resource Core (Boston, Mass.). The open reading frame was amplified by high fidelity PCR and transferred into the multiple cloning site of pCDH-CMV-MCS-EF1-copGFP. Integrity of the insert was confirmed by sequencing. Lentiviral particles were generated by cotransfecting the pCDH-FOXA1-GFP vector and lentiviral packaging constructs into HEK293T cells and allowing virus to accumulate in the medium for 48 h. Viral titer was estimated over a dilution series in AB32 cells, using a Facs Calibur flow cytometer to estimate GFP positivity. AB32 cells were infected at a level predicted to give a 70% infection rate and incubated for 24 h at 37° C. to allow expression of FOXA1, followed by treatment for 0, 6 and 24 h with 10 nM ORG2058. Matched control samples infected with the parent pCDH-CMV-MCS-EF1-copGFP virus were included at each time point.

### Example 18

### Gene Expression Microarray

Total RNA was isolated using RNAqueous purification columns (Invitrogen). Total RNA (500 ng) was amplified and biotin labelled using Illumina TotalPrep reagents (Invitrogen). The amplified samples (750 ng) were hybridized to human whole genome HT-12 gene expression bead arrays using the recommended Illumina reagents and following the manufacturer's protocol. Raw data were analysed using Genome Studio software (Illumina). After background subtraction data and cubic spline normalization, differential expression p values were determined using the Illumina custom model of Genome Studio. Data were further analysed using Microsoft Excel and SPSS statistical software. Hierarchical clustering and self organizing map clustering were performed using GenePattern [65].

### Example 19

### Protein Extract Preparation and Immunoblotting

Cells to be analyzed by protein immunoblot were harvested by trypsinization, washed with cold phosphate buffered saline solution and collected into a cell pellet by centrifugation. Whole cell extracts were prepared by lysis of cells in RIPA buffer (10 mM NaPO4 (pH 7.0), 150 mM NaCl, 2 mM EDTA, 1% sodium deoxycholate, 1% NP-40, 0.1% β-mercaptoethanol) containing 10 mM NaMoO4, 1% aprotinin, Complete protease inhibitor (Roche, Castle Hill, Australia) and 0.5 mM phenylmethylsulfonylfluoride, and rotation 15 min at 4° C. Insoluble debris was removed by centrifugation at 14,000×g, 15 min at 4° C. Protein concentration was estimated using Bradford dye reagent (Bio-Rad, Regents Park, Australia). Proteins were fractionated by electrophoresis through denaturing 7.5% polyacrylamide-SDS gel and transferred to nitrocellulose membrane as described previously [66]. For detection of FOXA1 expression T-47D whole cell extract was loaded at 100 μg per lane and transduced cell extracts at 10 μg per lane. FOXA1 was detected using a goat anti-human FOXA1 polyclonal antibody (Abcam ab5089, Sapphire Biosciences, Waterloo, Australia) at 1:800 dilution, and rabbit anti-goat horseradish peroxidase conjugated secondary antibody (Dako Cytomation, Kingsgrove, Australia). For detection of PR protein expression, whole cell extracts were loaded as indicated. PR was detected using hPRa6 and hPRa7 in-house mouse monoclonal antibodies (1:100 each) and goat anti-mouse horseradish peroxidase conjugated secondary antibody (Dako). Protein bands were visualized by chemiluminescent reaction using ECL reagents (Quantum Scientific, Murrarie, Australia) and exposure to film or imaging using a Kodak Image Station (Carestream Health, Richmond, Australia).

### Example 20

### Genomic Distribution of PR Binding Sites in T-47D and AB32 Cells

As shown in FIG. 1, progestin-dependent PR bound DNA fragments identified by ChIP-seq were aligned to the genome using Bowtie and peaks of binding were identified using ERANGE, with 0.27% FDR. (A and B) Location of PR binding regions relative to transcription start sites of RefSeq genes in (A) T-47D and (B) AB32 cells was determined using CisGenome. (C and D) PR binding region distribution by chromosome, ranked by gene number in (C) T-47D and (D) AB32 cells. (E and F) Percentage of progestin regulated genes with PR binding regions within a given distance from the TSS in (E) T-47D and (F) AB32 cells. Solid line—binding regions associated with up-regulated genes, dashed line—binding regions associated with down regulated genes. The median distances of up- and down-regulated genes to nearest TSSs are indicated.

### Example 21

### PR Binding Associated with Progestin Responsive Genes

As shown in FIG. 2, gene expression profiles in T-47D and AB32 cells were determined at 2, 6 and 24 h after treatment with 10 nM ORG2058. Transcripts that were significantly differentially expressed at 2, 6 and/or 24 h, compared to untreated controls, were considered progestin regulated and were compared with the list of PR binding regions in the same cell line. (A and B) Overlap between PR binding regions and progestin regulated genes in (A) T-47D and (B) AB32 cells. (C and D) PR binding regions within 10 kb or in the 5′-UTR of genes regulated at 2, 6 and 24 h in (C) T-47D and (D) AB32 cells. (E) Genes with patterns of progestin regulation in T-47D cells that grouped together were identified by SOM cluster analysis using Gene Pattern. Patterns of regulation are plotted as the mean log fold change relative to the untreated control. Error bars represent the standard error of the mean. (F) Mean distance to TSS of PR binding regions associated with each SOM cluster shown in (E).

### Example 22

### Differential PR Binding and Transcriptional Regulation in T-47D and AB32

In reference to FIG. 3A, PR binding regions that were common between T-47D and AB32 cells were identified using the IntersectBed function in Bed Tools. The number and percentage of regions that were unique to T-47D or AB32 cells or common to both cell lines are shown.

In reference to FIG. 3B, T-47D and AB32 cells were treated with 10 nM ORG2058 (ORG) or vehicle for 2, 6 and 24 h. Transcript expression was measured by whole genome microarray. Genes that were differentially expressed in ORG2058 treated samples relative to the untreated control at one or more time point in T-47D or AB32 cells were compared by unsupervised hierarchical cluster analysis. Red—increased expression, green—decreased expression relative to vehicle treated control.

In reference to FIG. 3C, overlap between transcripts regulated by progestins in T-47D and AB32 cells at 2, 6 or 24 h is shown. The numbers and percentage of transcripts that were uniquely regulated by progestins in T-47D or AB32 and regulation that was common to both cell lines are shown.

In reference to FIG. 3D, the numbers of progestin regulated transcripts in T-47D and AB32 at individual treatment times is shown. The number of transcripts uniquely regulated in T-47D or AB32 cells or regulated in both at a specific time point is shown.

### Example 23

### Motif Enrichment in PR Binding Regions

In reference to FIG. 4A, full length and core PREs identified in binding regions in T-47D and AB32 cells are shown. Regulation-associated PR binding region sequences were analysed for statistical enrichment of conserved sequence motifs using MEME-ChIP and Homer. Full and core PR binding elements were discovered in both cell lines. The full length PRE identified by MEME-ChIP and core element identified in Homer are shown.

In reference to FIGS. 4B and C, PRE strength by p value in (B) T-47D and (C) AB32 cells is shown. PREs were classified as strong (+++, p<1×10-5), moderate (++, p=1×10-5 to 1×10-3) or weak/absent (+, p>1×10-3).

In reference to FIGS. 4D and 4E, PR peak height in regulation and non-regulation associated PR binding regions in (D) T-47D and (E) AB32 cells is shown. (F) Top transcription factor binding motif enrichment in T-47D and AB32 cells.

### Example 24

### Introduction of FOXA1 into AB32 Cells Alters Progestin Response

In reference to FIG. 5A, unsupervised cluster analysis of transcriptional profiles in response to progestin in AB32 cells in the presence and absence of FOXA1 is shown. AB32 cells were transduced for 24 h with viral particles comprising the pCDH-FOXA1 construct or empty pCDH control. The cells were treated 6 and 24 h with 10 nM ORG2058 (ORG) or vehicle. Gene expression was measured by whole genome microarray. Genes that were differentially expressed at any time point in ORG2058 treated cells compared to control were analysed by unsupervised hierarchical cluster analysis. Red—increased log fold expression, green—decreased log fold expression.

In reference to FIG. 5B, FOXA1 protein expression in AB32 cells and parent MCF-10A cells before and after viral transduction, compared with endogenous expression in T-47D cells is shown.

In reference to FIG. 5C, the numbers of progestin regulated transcripts in AB32 cells in the presence and absence of FOXA1 is shown.

In reference to FIG. 5D, comparison of FOXA1 binding strength at PR, ER and FOXA1 binding sites is shown.

In reference to FIG. 5E, the ratio of PR binding regions to regulated genes in AB32 cells for genes that lost, gained or retained progestin regulation with FOXA1 expression is shown.

In reference to FIG. 5F, the distance from PR binding regions in AB32 to genes that lost, gained or retained progestin regulation with FOXA1 expression is shown. Error bars represent standard error of the mean.

### Example 26

### PR Binding Region to Chromosome Distribution in T-47D and AB32 Cells

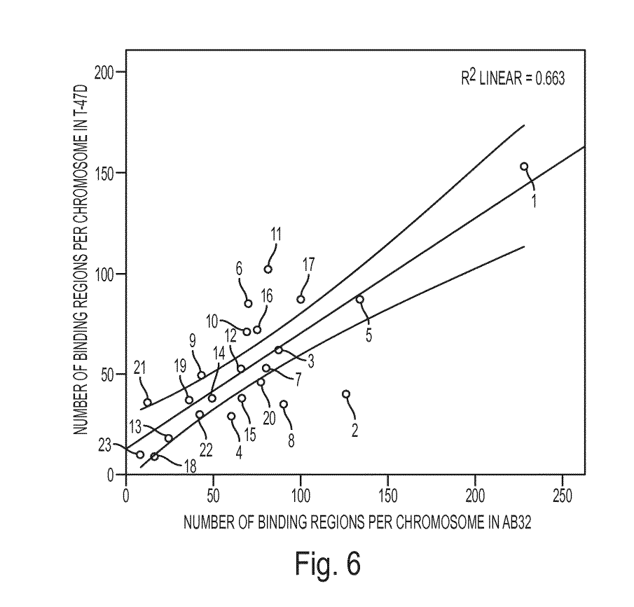

In reference to FIG. 6, the total numbers of PR binding region per chromosome were compared by linear regression between T-47D and AB32 datasets. Line of fit and 95% confidence intervals are shown. Labels represent chromosome number.

### Example 27

### Relationship Between PR Binding and Time of Progestin Regulation

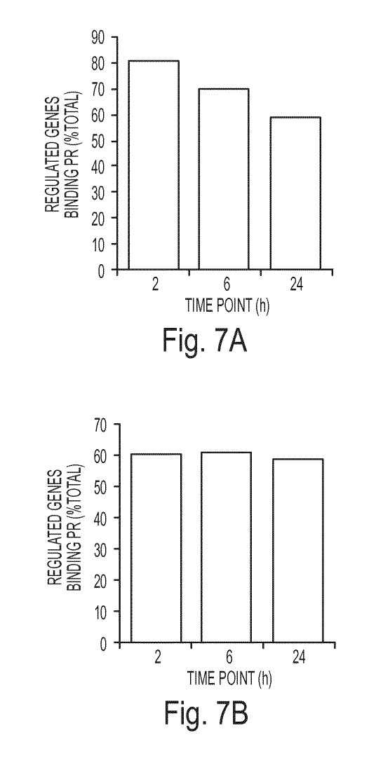

In reference to FIG. 7, the proportion of progestin regulated genes at 2, 6 or 24 h after treatment, which were associated with one or more PR binding regions was determined in (A) T-47D and (B) AB32 cells.

### Example 28

### Location of PR Binding Regions

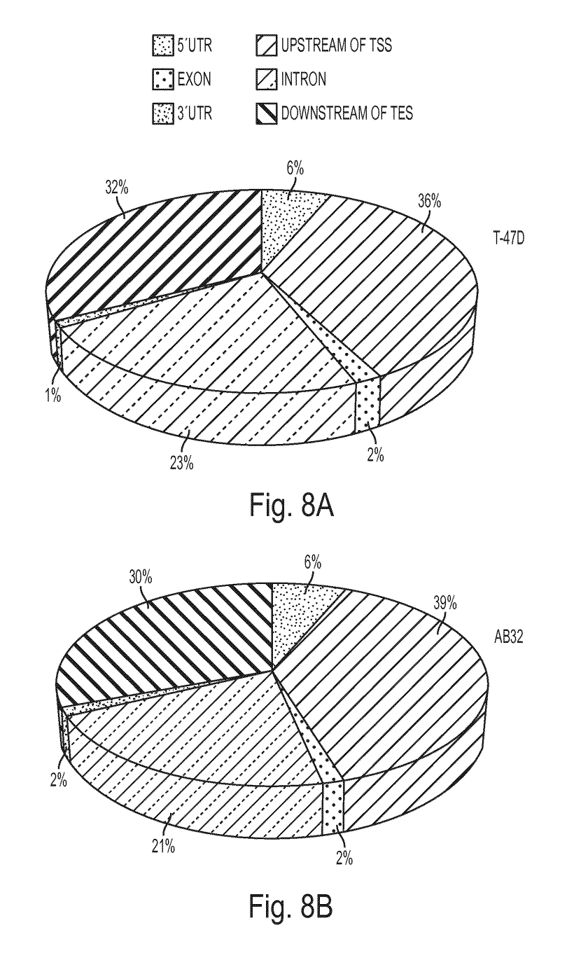

In reference to FIG. 8, the distribution of all PR binding regions, with respect to the nearest gene, was determined using CisGenome v1.1 in (A) T-47D and (B) AB32 cells.

### Example 29

### Location of PR Binding Regions

In reference to FIG. 9, the distribution of all PR binding regions, with respect to the nearest gene, was determined using CisGenome v1.1 in (A) T-47D and (B) AB32 cells.

### Example 30

### Patterns of Transcriptional Regulation in AB32 Cells

In reference to FIG. 10, progestin regulated transcripts were identified in AB32 cells at 2, 6 and 24 h treatment with 10 nM ORG2058 by gene expression profiling. Patterns of transcriptional regulation over the 24 h time course were identified by self-organizing map clustering using Gene Pattern.

### Example 31

### PR Binding Regions in T-47D and AB32 Cells

In reference to FIG. 11, examples of PR binding regions that were unique to T-47D or AB32 cells or common to both lines are shown as custom tracks displayed in the UCSC genome browser.

### Example 32

### Validation of Cell Type-Specific PR Binding Regions Identified in ChIP-seq

In reference to FIG. 12, PR binding regions identified in (A) T-47D, (B) AB32 or (C) both cell lines by ChIP-seq were validated by directed PR-ChIP, using binding region-specific primers and quantitative real-time PCR. Regions bound near ACSL1 and PACSIN1, which were regulated in T-47D but not AB32 produced marked enrichment of bound fragments in T-47D cells and not AB32. The converse was true with PR target regions identified in AB32 but not T-47D. FLRT3 and DHRS9, which are both transcriptional targets only in AB32, were strongly bound by PR in AB32 but showed a weak association in T-47D cells. PDK4 and Notch 2, which are progestin regulated in both cell lines, were bound by PR in both although the association was stronger in T-47D (85-fold vs 42-fold binding enrichment of PDK-4 and 300-fold vs 37-fold enrichment of Notch 2 binding in T-47D vsAB32).

### Example 33

### Overlap of PR Binding Regions in ORG2058-Treated T-47D and AB32 with Binding in T-47D after Progesterone (P4) Treatment (FIG. 13)

Our data are compared with progesterone-liganded PR binding in T-47D summarized in Tang et al [1] and available at http://cistrome.dfci.harvard.edu/NR_Cistrome/.

### Example 34

### Progestin Regulation of Gene Expression in Additional Breast Cell Lines

In reference to FIG. 14, ZR-75-1 breast cancer cells and AB9 PR-positive transformed normal breast cells were treated for 2, 6 or 24 h with 10 nM ORG2058 or vehicle, then harvested and total RNA was isolated. Gene expression levels were estimated by Illumina HT-12 microarray. Data were analyzed using Genome Studio software. Transcripts with levels that were significantly different in ORG2058 compared to vehicle-treated cells (diff p value <0.01) and had a fold change of 1.5 or more were considered progestin regulated. (A) Numbers of progestin regulated transcripts in ZR-75-1 or AB9 cells or both. (B) Unsupervised average linkage hierarchical cluster analysis of arrays (Pearson correlation) and gene expression fold change (uncentred correlation) was performed on the subset of transcripts that were progestin regulated in one or both cell lines, using Gene Pattern. Red—increased expression, green—decreased expression with ORG2058, relative to vehicle.

### Example 35

### PRE and Cofactor Motif Enrichment in Regulation-Associated Binding Sites in T-47D and AB32 Cells

In reference to FIG. 15, the relative proportions of regulation-associated PR binding regions containing PREs with or without one or more of the top enriched transcriptional cofactor binding motifs are shown. (A) T-47D and (B) AB32 motif distribution are shown.

### Example 36

### Distribution of PRE Position in PR Binding Regions in T-47D and AB32 Cells

In reference to FIG. 16, the positions of PRE motifs in PR binding regions relative to peak center are plotted as a frequency distribution in (A) T-47D and (B) AB32 cells.

### Example 37

### PRE Strength does not Predict PR Binding

In reference to FIG. 17, PRE motifs were classified in PR binding regions using the FIMO program in MEME [2]. The strength of the strongest candidate PRE, as determined by p value, in each binding region was plotted against peak height, as an indicator of PR binding strength. Estimated line of fit and Pearson correlation R² value were estimated. Data are shown for (A) T-47D and (B) AB32 cells.

### Example 38

### FOXA1 Transcript Expression in Cell Lines

In reference to FIG. 18, FOXA1 transcript expression, measured on Illumina HT-12 arrays, was compared in breast cancer (T-47D, ZR-75-1) and transformed normal breast (AB9, AB32) cells. FOXA1 levels are expressed relative to the level in AB32 cells.

### Example 39

### FOXA1 Binding at PR Binding Regions with or without Predicted FOXA1 Motifs

In reference to FIG. 19, the presence of FOXA1 motifs in PR binding regions was predicted using Homer software. PR binding regions predicted to bind FOXA1 and regions lacking FOXA1 binding motifs were separately analyzed for actual FOXA1 binding enrichment. Average FOXA1 binding strength in T-47D from ChIP-seq is shown at PR binding regions containing FOXA1 motifs (blue line) and in PR binding regions that lacked any predicted FOXA1 motif (red line).

### Example 40

### PR Expression in T-47D, AB32 and AB9 Cells

In reference to FIG. 20, proteins from whole cell extracts at the loading indicated were fractionated by denaturing 7.5% polyacrylamide-SDS gel electrophoresis and transferred to nitrocellulose membrane. PR protein bands were visualized as described in herein.

### Example 41

### Chromosome in AB32

TABLE S1
Summary of gene functional annotation by Gene ontology
term enrichment was determined for the subset of PR binding
region-associated genes in AB32 cells on chromosomes 2, 8 and 11.

| | Enrichment Score | Function |
|---|---|---|
| Chr 2 (58 IDs) | | |
| Annotation Cluster | | |
| 1 | 2.75 | Metabolism |
| 2 | 1.61 | GTPase activity |
| 3 | 0.91 | Metabolism |
| Chr 8 (25 IDs) | | |
| Annotation Cluster | | |
| 1 | 1.62 | Apoptosis |
| 4 | 0.72 | Catabolism |
| 6 | 0.55 | Transcriptional regulation |
| 7 | 0.52 | Apoptosis |
| 8 | 0.39 | Nucleotide binding |
| 11 | 0.11 | Metal ion binding |
| Chr 11 (39 IDs) | | |
| Annotation Cluster | | |
| 1 | 1.57 | Metabolism |
| 3 | 0.73 | DNA repair |
| 4 | 0.72 | Protein complex assembly |
| 5 | 0.70 | Transcriptional regulation |
| 6 | 0.63 | Phosphorylation |
| 7 | 0.51 | Transcriptional regulation |
| 8 | 0.37 | Apoptosis |

### Example 42

TABLE S2
Validation of top PR binding regions
PR binding to selected sites in T-47D and AB32 cells was confirmed by directed
ChIP-PCR. Ten binding regions were selected for validation in T-47D and nine in AB32 cells.
T-47D

| | | | | | | | Validation |
|---|---|---|---|---|---|---|---|
| | Region position | | | Peak | PRE | PRE | Fold |
| Region ID | chromosome | start | end | Height | p-value | score | enrichment |
| T47D4122 | chr3 | 184316710 | 184317034 | 40.7 | 3.96E−05 | ++ | 3.97 |
| T47D6084 | chr9 | 122679651 | 122680502 | 26.2 | 2.55E−06 | +++ | 5.68 |
| T47D6046 | chr9 | 111991161 | 111991613 | 24.1 | 7.76E−04 | ++ | 46.53 |
| T47D1380 | chr11 | 128699096 | 128700220 | 20.9 | 2.07E−04 | ++ | 4.08 |
| T47D2524 | chr16 | 84310655 | 84311589 | 20.8 | 4.35E−04 | ++ | 1.29 |
| T47D283 | chr1 | 120376407 | 120376763 | 20.2 | 6.29E−04 | ++ | 7.14 |
| T47D3464 | chr20 | 44776235 | 44776939 | 20.1 | 1.24E−04 | ++ | 12.27 |
| T47D5837 | chr8 | 102519793 | 102520361 | 19.1 | 2.03E−03 | + | 13.06 |
| T47D3514 | chr20 | 57834740 | 57835410 | 14.6 | 8.84E−07 | +++ | 4.34 |
| T47D4818 | chr5 | 148845545 | 148845884 | 12.9 | 1.20E−04 | ++ | 13.29 |

AB32

| | | | | | | | Validation |
|---|---|---|---|---|---|---|---|
| | Region position | | | Peak | PRE | PRE | Fold |
| Region ID | chromosome | start | end | Height | p-value | score | enrichment |
| AB32399 | chr1 | 143957617 | 143957902 | 13.2 | 6.32E−07 | +++ | 6.49 |
| AB326156 | chr5 | 141702092 | 141702799 | 9.2 | 7.33E−04 | ++ | 2.01 |
| AB326110 | chr5 | 134596597 | 134597211 | 8.4 | 3.69E−06 | +++ | 16.08 |
| AB324311 | chr2 | 223025077 | 223026178 | 7.9 | 1.38E−05 | ++ | 9.92 |
| AB324058 | chr2 | 114040782 | 114041064 | 7.8 | 3.66E−04 | ++ | 1.24 |
| AB32833 | chr1 | 227827184 | 227827908 | 7.8 | 7.33E−04 | ++ | 6.28 |
| AB323972 | chr2 | 91137645 | 91137989 | 7.8 | 6.73E−05 | ++ | 2.86 |
| AB324554 | chr20 | 33358266 | 33358837 | 7.2 | 3.51E−08 | +++ | 0.91 |
| AB32810 | chr1 | 223989877 | 223991355 | 7.2 | 5.12E−04 | ++ | 2.21 |

### Example 43

TABLE S3
Functional analysis of progestin regulated gene clusters
lost, gained and conserved with expression of FOXA1.
Functional annotation clustering was performed for
the groups of genes that lost, gained and conserved
progestin regulation in AB32 cells after expression of FOXA1.

| | Enrichement |
|---|---|
| Functional group | Score |
| Lost | |
| Apoptosis | 6.68 |
| Steroid biosynthesis | 2.90 |
| Regulation of protein kinase | 2.41 |
| activity | |
| Protein transport | 2.17 |
| Gained | |
| Blood vessel morphogenesis | 3.28 |
| Cell motility | 2.03 |
| Conserved | |
| Mitotic cell cycle | 4.37 |
| Wound healing | 3.55 |
| Apoptosis | 2.40 |

Although the above description refers to particular aspects, it is to be understood that these aspects are merely illustrative. It will be apparent to those skilled in the art that various modifications and variations can be made to the polymorphic forms and methods described herein. Thus, it is intended that the present description include modifications and variations that are within the scope of the description and their equivalents.

### REFERENCES

The references cited herein are incorporated by reference in their entirety.
1. Hubler T R, Scammell J G (2004) Intronic hormone response elements mediate regulation of FKBP5 by progestins and glucocorticoids. Cell Stress Chaperones 9: 243-252.
2. Lieberman B A, Bona B J, Edwards D P, Nordeen S K (1993) The constitution of a progesterone response element. Mol Endocrinol 7: 515-527.
3. Nelson C C, Hendy S C, Shukin R J, Cheng H, Bruchovsky N, et al. (1999) Determinants of DNA sequence specificity of the androgen, progesterone, and glucocorticoid receptors: evidence for differential steroid receptor response elements. Mol Endocrinol 13: 2090-2107.
4. Onate S A, Tsai S Y, Tsai M J, O'Malley B W (1995) Sequence and characterization of a coactivator for the steroid hormone receptor superfamily. Science 270: 1354-1357.
5. Liu Z, Wong J, Tsai S Y, Tsai M J, O'Malley B W (2001) Sequential recruitment of steroid receptor coactivator-1 (SRC-1) and p300 enhances progesterone receptor-dependent initiation and reinitiation of transcription from chromatin. Proc Natl Acad Sci USA 98: 12426-12431.
6. McKenna N J, O'Malley B W (2002) Combinatorial control of gene expression by nuclear receptors and coregulators. Cell 108: 465-474.
7. Scarpin K M, Graham J D, Mote P A, Clarke C L (2009) Progesterone action in human tissues: regulation by progesterone receptor (PR) isoform expression, nuclear positioning and coregulator expression. Nucl Recept Signal 7: e009.
8. Vicent G P, Zaurin R, Nacht A S, Li A, Font-Mateu J, et al. (2009) Two chromatin remodeling activities cooperate during activation of hormone responsive promoters. PLoS Genet. 5: e1000567.
9. Vicent G P, Ballare C, Zaurin R, Saragueta P, Beato M (2006) Chromatin remodeling and control of cell proliferation by progestins via cross talk of progesterone receptor with the estrogen receptors and kinase signaling pathways. Ann N Y Acad Sci 1089: 59-72.
10. Vicent G P, Nacht A S, Zaurin R, Ballare C, Clausell J, et al. (2010) Minireview: role of kinases and chromatin remodeling in progesterone signaling to chromatin. Mol Endocrinol 24: 2088-2098.
11. Vicent G P, Zaurin R, Nacht A S, Font-Mateu J, Le Dily F, et al. (2010) Nuclear factor 1 synergizes with progesterone receptor on the mouse mammary tumor virus promoter wrapped around a histone H3/H4 tetramer by facilitating access to the central hormone-responsive elements. J Biol Chem 285: 2622-2631.
12. Friedman J R, Kaestner K H (2006) The Foxa family of transcription factors in development and metabolism. Cell Mol Life Sci 63: 2317-2328.
13. Bernardo G M, Lozada K L, Miedler J D, Harburg G, Hewitt S C, et al. (2010) FOXA1 is an essential determinant of ERalpha expression and mammary ductal morphogenesis. Development 137: 2045-2054.
14. Carroll J S, Meyer C A, Song J, Li W, Geistlinger T R, et al. (2006) Genome-wide analysis of estrogen receptor binding sites. Nat Genet. 38: 1289-1297.
15. Hurtado A, Holmes K A, Ross-Innes C S, Schmidt D, Carroll J S (2011) FOXA1 is a key determinant of estrogen receptor function and endocrine response. Nat Genet. 43: 27-33.
16. Lupien M, Eeckhoute J, Meyer C A, Wang Q, Zhang Y, et al. (2008) FoxA1 translates epigenetic signatures into enhancer-driven lineage-specific transcription. Cell 132: 958-970.
17. Wang Q, Li W, Liu X S, Carroll J S, Janne O A, et al. (2007) A hierarchical network of transcription factors governs androgen receptor-dependent prostate cancer growth. Mol Cell 27: 380-392.
18. Cicatiello L, Addeo R, Sasso A, Altucci L, Petrizzi V B, et al. (2004) Estrogens and progesterone promote persistent CCND1 gene activation during G1 by inducing transcriptional derepression via c-Jun/c-Fos/estrogen receptor (progesterone receptor) complex assembly to a distal regulatory element and recruitment of cyclin D1 to its own gene promoter. Mol Cell Biol 24: 7260-7274.
19. Kushner P J, Agard D A, Greene G L, Scanlan T S, Shiau A K, et al. (2000) Estrogen receptor pathways to AP-1. J Steroid Biochem Mol Biol 74: 311-317.
20. Tseng L, Tang M, Wang Z, Mazella J (2003) Progesterone receptor (hPR) upregulates the fibronectin promoter activity in human decidual fibroblasts. DNA Cell Biol 22: 633-640.
21. Beguelin W, Diaz Flaque M C, Proietti C J, Cayrol F, Rivas M A, et al. (2010) Progesterone receptor induces ErbB-2 nuclear translocation to promote breast cancer growth via a novel transcriptional effect: ErbB-2 function as a coactivator of Stat3. Mol Cell Biol 30: 5456-5472.
22. Faivre E J, Daniel A R, Hillard C J, Lange C A (2008) Progesterone receptor rapid signaling mediates serine 345 phosphorylation and tethering to specificity protein 1 transcription factors. Mol Endocrinol 22: 823-837.
23. Graham J D, Clarke C L (1997) Physiological action of progesterone in target tissues. Endocr Rev 18: 502-519.
24. Clarke C L, Sutherland R L (1990) Progestin regulation of cellular proliferation. Endocr Rev 11: 266-301.
25. Longacre T A, Bartow S A (1986) A correlative morphologic study of human breast and endometrium in the menstrual cycle. Am J Surg Pathol 10: 382-393.
26. Ace C I, Okulicz W C (2004) Microarray profiling of progesterone-regulated endometrial genes during the rhesus monkey secretory phase. Reprod Biol Endocrinol 2: 54.
27. Borthwick J M, Charnock-Jones D S, Tom B D, Hull M L, Teirney R, et al. (2003) Determination of the transcript profile of human endometrium. Mol Hum Reprod 9: 19-33.
28. Graham J D, Yager M L, Hill H D, Byth K, O'Neill G M, et al. (2005) Altered progesterone receptor isoform expression remodels progestin responsiveness of breast cancer cells. Mol Endocrinol 19: 2713-2735.
29. Kao L C, Tulac S, Lobo S, Imani B, Yang J P, et al. (2002) Global gene profiling in human endometrium during the window of implantation. Endocrinology 143: 2119-2138.
30. Richer J K, Jacobsen B M, Manning N G, Abel M G, Wolf D M, et al. (2002) Differential gene regulation by the two progesterone receptor isoforms in human breast cancer cells. J Biol Chem 277: 5209-5218.
31. Beral V, Million Women Study C (2003) Breast cancer and hormone-replacement therapy in the Million Women Study. Lancet 362: 419-427.
32. Beral V, Reeves G, Bull D, Green J Breast Cancer Risk in Relation to the Interval Between Menopause and Starting Hormone Therapy. J Natl Cancer Inst 103: 296-305.
33. Rossouw J E, Anderson G L, Prentice R L, LaCroix A Z, Kooperberg C, et al. (2002) Risks and benefits of estrogen plus progestin in healthy postmenopausal women: principal results From the Women's Health Initiative randomized controlled trial. JAMA 288: 321-333.
34. Beral V, Reeves G, Bull D, Green J (2011) Breast cancer risk in relation to the interval between menopause and starting hormone therapy. J Natl Cancer Inst 103: 296-305.
35. Chlebowski R T, Anderson G L, Gass M, Lane D S, Aragaki A K, et al. (2010) Estrogen plus progestin and breast cancer incidence and mortality in postmenopausal women. JAMA 304: 1684-1692.
36. Graham J D, Mote P A, Salagame U, van Dijk J H, Balleine R L, et al. (2009) DNA replication licensing and progenitor numbers are increased by progesterone in normal human breast. Endocrinology 150: 3318-3326.
37. Langmead B, Trapnell C, Pop M, Salzberg S L (2009) Ultrafast and memory-efficient alignment of short DNA sequences to the human genome. Genome Biol 10: R25.
38. Mortazavi A, Williams B A, McCue K, Schaeffer L, Wold B (2008) Mapping and quantifying mammalian transcriptomes by RNA-Seq. Nat. Methods 5: 621-628.
39. Roschke A V, Tonon G, Gehlhaus K S, McTyre N, Bussey K J, et al. (2003) Karyotypic complexity of the NCI-60 drug-screening panel. Cancer Res 63: 8634-8647.
40. Tang Q, Chen Y, Meyer C, Geistlinger T, Lupien M, et al. (2011) A comprehensive view of nuclear receptor cancer cistromes. Cancer Res 71: 6940-6947.
41. Wang D, Garcia-Bassets I, Benner C, Li W, Su X, et al. (2011) Reprogramming transcription by distinct classes of enhancers functionally defined by eRNA. Nature 474: 390-394.
42. Joseph R, Orlov Y L, Huss M, Sun W, Kong S L, et al. (2010) Integrative model of genomic factors for determining binding site selection by estrogen receptor-alpha. Mol Syst Biol 6: 456.
43. Bravieri R, Shiyanova T, Chen T H, Overdier D, Liao X (1997) Different DNA contact schemes are used by two winged helix proteins to recognize a DNA binding sequence. Nucleic Acids Res 25: 2888-2896.
44. Cirillo L A, Zaret K S (2007) Specific interactions of the wing domains of FOXA1 transcription factor with DNA. J Mol Biol 366: 720-724.
45. Pierrou S, Hellqvist M, Samuelsson L, Enerback S, Carlsson P (1994) Cloning and characterization of seven human forkhead proteins: binding site specificity and DNA bending. EMBO J. 13: 5002-5012.
46. Reddy T E, Pauli F, Sprouse R O, Neff N F, Newberry K M, et al. (2009) Genomic determination of the glucocorticoid response reveals unexpected mechanisms of gene regulation. Genome Res 19: 2163-2171.
47. Welboren W J, van Driel M A, Janssen-Megens E M, van Heeringen S J, Sweep F C, et al. (2009) ChIP-Seq of ERalpha and RNA polymerase II defines genes differentially responding to ligands. EMBO J. 28: 1418-1428.
48. Macquarrie K L, Fong A P, Morse R H, Tapscott S J (2011) Genome-wide transcription factor binding: beyond direct target regulation. Trends Genetics 27: 141-148.
49. Krum S A, Miranda-Carboni G A, Lupien M, Eeckhoute J, Carroll J S, et al. (2008) Unique ERalpha cistromes control cell type-specific gene regulation. Molecular Endocrinology 22: 2393-2406.
50. So A Y, Chaivorapol C, Bolton E C, Li H, Yamamoto K R (2007) Determinants of cell- and gene-specific transcriptional regulation by the glucocorticoid receptor. PLoS Genet. 3: e94.
51. Bolton E C, So A Y, Chaivorapol C, Haqq C M, Li H, et al. (2007) Cell- and gene-specific regulation of primary target genes by the androgen receptor. Genes Dev 21: 2005-2017.
52. Yin P, Roqueiro D, Huang L, Owen J K, Xie A, et al. (2012) Genome-wide progesterone receptor binding: cell type-specific and shared mechanisms in T47D breast cancer cells and primary leiomyoma cells. PLoS One 7: e29021.
53. Rayasam G V, Elbi C, Walker D A, Wolford R, Fletcher T M, et al. (2005) Ligand-specific dynamics of the progesterone receptor in living cells and during chromatin remodeling in vitro. Mol Cell Biol 25: 2406-2418.
54. Belikov S, Astrand C, Wrange O (2009) FoxA1 binding directs chromatin structure and the functional response of a glucocorticoid receptor-regulated promoter. Mol Cell Biol 29: 5413-5425.
55. Garcia-Bassets I, Kwon Y-S, Telese F, Prefontaine G G, Hutt K R, et al. (2007) Histone methylation-dependent mechanisms impose ligand dependency for gene activation by nuclear receptors. Cell 128: 505-518.
56. Metzger E, Wissmann M, Yin N, Muller J M, Schneider R, et al. (2005) LSD1 demethylates repressive histone marks to promote androgen-receptor-dependent transcription. Nature 437: 436-439.
57. John S, Sabo P J, Thurman R E, Sung M H, Biddie S C, et al. (2011) Chromatin accessibility pre-determines glucocorticoid receptor binding patterns. Nat Genet. 43: 264-268.
58. Murtagh J, Martin F, Gronostajski R M (2003) The Nuclear Factor I (NFI) gene family in mammary gland development and function. J Mammary Gland Biol Neoplasia 8: 241-254.
59. Streuli C H, Edwards G M, Delcommenne M, Whitelaw C B, Burdon T G, et al. (1995) Stat5 as a target for regulation by extracellular matrix. J Biol Chem 270: 21639-21644.
60. Clarke C L, Zaino R J, Feil P D, Miller J V, Steck M E, et al. (1987) Monoclonal antibodies to human progesterone receptor: characterization by biochemical and immunohistochemical techniques. Endocrinology 121: 1123-1132.
61. Ji H, Jiang H, Ma W, Johnson D S, Myers R M, et al. (2008) An integrated software system for analyzing ChIP-chip and ChIP-seq data. Nat Biotechnol 26: 1293-1300.
62. Heinz S, Benner C, Spann N, Bertolino E, Lin Y C, et al. (2010) Simple combinations of lineage-determining transcription factors prime cis-regulatory elements required for macrophage and B cell identities. Mol Cell 38: 576-589.
63. Bailey T L, Williams N, Misleh C, Li W W (2006) MEME: discovering and analyzing DNA and protein sequence motifs. Nucleic Acids Res 34: W369-373.
64. Bailey T L, Boden M, Whitington T, Machanick P (2010) The value of position-specific priors in motif discovery using MEME. BMC Bioinformatics 11: 179.
65. Reich M, Liefeld T, Gould J, Lerner J, Tamayo P, et al. (2006) GenePattern 2.0. Nat Genet. 38: 500-501.
66. Graham J D, Yeates C, Balleine R L, Harvey S S, Milliken J S, et al. (1995) Characterization of progesterone receptor A and B expression in human breast cancer. Cancer Res 55: 5063-5068.
67. Tang Q, Chen Y, Meyer C, Geistlinger T, Lupien M, et al. (2011) A comprehensive view of nuclear receptor cancer cistromes. Cancer Res 71: 6940-6947.
68. Bailey T L, Williams N, Misleh C, Li W W (2006) MEME: discovering and analyzing DNA and protein sequence motifs. Nucleic Acids Res 34: W369-373.

### ABBREVIATIONS

### Incorporated by Reference Herein in their Entirety

AP-1 Activator protein 1
AR Androgen receptor
Bowtie Sequence alignment software (http://bowtie-bio.sourceforge.net)
CD44 cell surface glycoprotein CD44
ChIP Chromatin immunoprecipitation
CTCF CCCTC-binding factor
ER Estrogen receptor
ERANGE findall.py ChIP peak identification software tool (http://woldlab.caltech.edu/rnaseq)
FDR False discovery rate
FOXA1 Forkhead box A1
GR Glucocorticoid receptor
Homer Sequence analysis software for ChIP peak identification and DNA binding motif identification (http://biowhat.ucsd.edu/homer/)
MEME-ChIP Sequence analysis online tool for DNA binding motif identification specifically in ChIP-seq data (http://meme.nbcr.net/)
NF1 Nuclear factor 1
ORG Organon 2058
PRE Progesterone response element
SOM Self-organizing map
SP1 Specificity protein 1
Stat3 Signal transducer and activator of transcription
TSS Transcription start site

What is claimed as new and desired to be protected by Letters Patent of the United States is:

1. A method of treating a patient with an anti-progestin, comprising:
obtaining a tissue sample suspected of being tumorigenic or cancerous from a patient;
binding the progesterone receptor to genomic DNA in the tissue sample;
detecting genomic DNA associated with the progesterone receptor in the tissue sample;
detecting the level of one or more activated progesterone receptor associated DNA targets in the tissue sample and in a negative control sample; and
administering an anti-progestin to the patient if the level of the one or more activated progesterone receptor associated DNA targets is at least about 4-fold greater than the level in the negative control sample or statistically significantly greater than the level in the negative control sample.

2. The method of claim 1, wherein the progesterone receptor is bound to genomic DNA in the tissue sample by cross-linking.

3. The method of claim 1, wherein the one or more activated progesterone receptor associated DNA targets comprise progesterone receptor binding regions near to ACSL1 and PACSIN1.

4. The method of claim 3, wherein the one or more activated progesterone receptor associated DNA targets are detected using oligonucleotide primer pairs directed to progesterone receptor binding regions near to ACSL1 and PACSIN1.

5. The method of claim 1, wherein the tissue sample is selected from group consisting of breast, brain, meningiomas, prostate, ovarian, endometrial, uterine sarcomas, uterine leiomyoma and lung tissue.

6. The method of claim 1, wherein the anti-progestin is selected from the group consisting of onapristone, lonaprisan, mifepristone, PF-02413873, telapristone, lilopristone, ORG2058, apoprisnil, ulipristal, ZM172406, ZM150271, ZM172405 and aglepristone.

7. The method of claim 1, wherein the anti-progestin is administered to the patient in an amount from about 10 mg to about 200 mg per day.

8. The method of claim 1, further comprising administering an anti-tumor compound.

9. The method of claim 8, wherein the anti-tumor compound is selected from the group consisting of everolimus, trastuzumab, TM1-D, anti-HER2 drugs, bevacizumab, paclitaxel, docetaxel, taxanes, doxorubicin, liposomal doxorubicin, pegylated liposomal doxorubicin, anthracyclines, anthracenediones, carboplatin, cisplatin, 5-FU, gemcitabine, cyclophosphamide, anti-estrogen, selective estrogen receptor modulators, aromatase inhibitors, and anti-androgens.

10. The method of claim 1, wherein the genomic DNA is detected by immunoprecipitation.

11. The method of claim 10, wherein the immunoprecipitation is performed using monoclonal antibodies hPRa6 and hPRa7.

12. The method of claim 1, wherein the progesterone receptor associated with genomic DNA is detected by immunoprecipitation with an anti-PR primary antibody.

13. A method of inhibiting the growth of a tumor susceptible to growth inhibition by anti-progestins, comprising:
obtaining a tissue sample suspected of being tumorigenic or cancerous from the tumor of a patient;
binding the progesterone receptor to genomic DNA in the tissue sample;
detecting genomic DNA associated with the progesterone receptor in the tissue sample;
detecting the level of one or more activated progesterone receptor associated DNA targets in the tissue sample and in a negative control sample; and
administering an anti-progestin to the patient if the level of the one or more activated progesterone receptor associated DNA targets is at least about 4-fold greater than the level in the negative control sample or is statistically significantly greater than the level in the negative control sample.

14. The method of claim 13, wherein the progesterone receptor is bound to genomic DNA in the tissue sample by cross-linking.

15. The method of claim 13, wherein the one or more activated progesterone receptor associated DNA targets comprise progesterone receptor binding regions near to ACSL1 and PACSIN1.

16. The method of claim 15, wherein the one or more activated progesterone receptor associated DNA targets are detected using oligonucleotide primer pairs directed to progesterone receptor binding regions near to ACSL1 and PACSIN1.

17. The method of claim 13, wherein the tumor is selected from group consisting of breast, brain, meningiomas, prostate, ovarian, endometrial, uterine sarcomas, uterine leiomyoma and lung tumors.

18. The method of claim 13, wherein the anti-progestin is selected from the group consisting of onapristone, lonaprisan, mifepristone, PF-02413873, telapristone, lilopristone, ORG2058, apoprisnil, ulipristal, ZM172406, ZM150271, ZM172405 and aglepristone.

19. The method of claim 13, wherein the anti-progestin is administered to the patient in an amount from about 10 mg to about 200 mg per day.

20. The method of claim 13, further comprising administering an anti-tumor compound.

21. The method of claim 20, wherein the anti-tumor compound is selected from the group consisting of everolimus, trastuzumab, TM1-D, anti-HER2 drugs, bevacizumab, paclitaxel, docetaxel, taxanes, doxorubicin, liposomal doxorubicin, pegylated liposomal doxorubicin, anthracyclines, anthracenediones, carboplatin, cisplatin, 5-FU, gemcitabine, cyclophosphamide, anti-estrogen, selective estrogen receptor modulators, aromatase inhibitors, and anti-androgens.

22. The method of claim 13, wherein the genomic DNA is detected by immunoprecipitation.

23. The method of claim 22, wherein the immunoprecipitation is performed using monoclonal antibodies hPRa6 and hPRa7.

24. The method of claim 13, wherein the progesterone receptor associated with genomic DNA is detected by immunoprecipitation with an anti-PR primary antibody.
